(19) United States
(12) Patent Application Publication
(10) Pub. No.: US 2022/0265210 A1
(43) Pub. Date: Aug. 25, 2022

(54) INFUSION DEVICE FOR CONTINUOUS GLUCOSE MONITORING

(71) Applicant: Pacific Diabetes Technologies Inc, Portland, OR (US)

(72) Inventors: Robert S. Cargill, Portland, OR (US); Solomon Reid, Portland, OR (US); Sheila Benware, Portland, OR (US); Chad Knutsen, Portland, OR (US); Andrew Greenberg, Portland, OR (US); Thomas Seidl, Portland, OR (US); William Kenneth Ward, Portland, OR (US)

(21) Appl. No.: 17/549,232

(22) Filed: Dec. 13, 2021

Related U.S. Application Data
(63) Continuation of application No. PCT/US2020/037511, filed on Jun. 12, 2020.
(60) Provisional application No. 62/861,940, filed on Jun. 14, 2019.

(51) Int. Cl. A61B 5/00; A61M 5/158; A61M 5/172; A61B 5/1486; A61B 5/145; A61M 5/142

(52) U.S. Cl. CPC A61B 5/4839; A61M 5/158; A61M 5/1723; A61B 5/14865; A61B 5/14532; A61M 5/142; A61M 2230/201

(57) ABSTRACT

The present disclosure provides systems and devices for combining analyte monitoring with fluid delivery, including devices that are adapted for use with combined sensors and cannulas having sensors and cannulas on a single component. These systems and devices may be used in various applications with simultaneous in vivo monitoring of analyte concentrations and delivery of medications.

### CROSS-REFERENCE

This application is a continuation of International Application No. PCT/US2020/037511, filed Jun. 12, 2020, which claims the benefit of U.S. Provisional Patent Application No. 62/861,940, filed Jun. 14, 2019, each of which is incorporated by reference herein in its entirety.

### BACKGROUND

Amperometric analyte sensors may be used to detect various analytes as oxygen, pH, glucose, lactate, drug metabolites, and pathogens in vivo. Further, sensors for Continuous Glucose Monitoring (CGM) may have widespread clinical adoption. These CGM sensors may reside in the subcutaneous tissue, and generate small glucose-dependent electrical currents that are detected by associated electronics. In many instances, it is desirable to both track the concentration of an analyte and deliver a medication in response to the level of the analyte. For example, this may be performed in the case of glucose analyte monitoring and insulin medication delivery, as insulin pumps may feature automated insulin dosing based upon readings from a CGM sensor.

### SUMMARY

The present disclosure provides devices and systems that use a combined sensor and cannula attached to a body that provides electrical coupling of the sensor to a signal processing device and fluidic coupling of the cannula to a medication delivery source, in order to combine subcutaneous liquid medication delivery and amperometric analyte sensing without a need for multiple skin piercing elements.

In an aspect, the present disclosure provides a device configured to perform simultaneous sensing of a concentration of an analyte and administration of a therapeutic fluid, comprising: a body comprising an upper housing, a lower housing, and a bottom, skin-contacting base, wherein the upper housing comprises a top face comprising a port configured to reversibly attach to a fluid delivery device configured for delivery of a fluid via insertion of a needle, wherein the port comprises a visible opening comprising a self-sealing septum in contact with the lower housing thereby forming an internal cavity; a sensing cannula comprising a proximal end, a distal end, an external surface, an internal lumen, at least one hollow channel within the internal lumen extending from the proximal end of the sensing cannula to the distal end of the sensing cannula configured for the administration of the therapeutic fluid, at least one indicating electrode on the external surface configured to sense the concentration of the analyte, and a conductor on the external surface extending from the proximal end of the sensing cannula to the at least one indicating electrode, wherein the proximal end of the sensing cannula is retained within the body, and wherein the distal end of the sensing cannula extends from the skin-contacting base; a channel within the body in fluid communication with the internal cavity formed by the self-sealing septum and the proximal end of the combined sensing cannula; a signal processing module, comprising a second body comprising an upper face, a lower face, and a vertical surface between the upper face and the lower face, wherein the vertical surface provides an electrical potential to the sensing cannula and receives an electrical current from the sensing cannula via a set of electrical contacts on the vertical surface, wherein the second body comprises a set of arms in contact with the upper housing, and wherein the lower face is in contact with the skin-contacting base; and an interface circuit comprising a proximal end and a distal end, wherein the interface circuit comprises one or more conductors configured to convey current signals from the sensing cannula to the signal processing module, wherein the proximal end of the interface circuit is in electrical contact with the proximal end of the sensing cannula, and wherein the distal end of the interface circuit is in electrical contact with the signal processing module.

In some embodiments, the fluid delivery device comprises a syringe or a pen. In some embodiments, the fluid delivery device comprises a syringe. In some embodiments, the fluid delivery device comprises a pen. In some embodiments, the at least one indicating electrode comprises an enzyme layer overlaying a conductive surface. In some embodiments, the enzyme layer is covered with a semi-permeable membrane. In some embodiments, the enzyme layer comprises glucose oxidase or glucose dehydrogenase. In some embodiments, the enzyme layer comprises an osmium-based redox mediator. In some embodiments, the osmium-based redox mediator comprises osmium dimethyl bipyridine. In some embodiments, the enzyme layer comprises polyvinylimidazole. In some embodiments, the sensing cannula comprises a reference electrode comprising silver/silver chloride (Ag/AgCl). In some embodiments, the signal processing module provides a bias potential to the sensing cannula of less than 250 millivolts (mV) relative to a reference potential. In some embodiments, the channel comprises a stainless steel needle connecting from the cavity to the proximal end of the sensing cannula. In some embodiments, the upper housing and the lower housing are configured to receive a hollow inserter needle partially enclosing the sensing cannula for insertion into a skin surface of a mammal. In some embodiments, the sensing cannula comprises a stiffness sufficient for insertion into a skin surface of a mammal without using an inserter needle. In some embodiments, the skin-contacting base comprises an adhesive surface configured to attach the device to a skin surface of a subject. In some embodiments, the analyte is selected from the group consisting of: oxygen, glucose, lactate, a drug metabolite, and a pathogen. In some embodiments, the analyte is glucose. In some embodiments, the therapeutic fluid is selected from the group consisting of: an insulin or insulin analog formulation, glatiramer acetate, heparin, human menopausal gonadotropin, vitamins, and minerals. In some embodiments, the therapeutic fluid is the insulin or the insulin analog formulation. In some embodiments, the insulin or the insulin analog formulation comprises an excipient comprising a phenol or cresol.

In another aspect, the present disclosure provides a device configured to perform simultaneous sensing of a concentration of an analyte and administration of a therapeutic fluid, comprising: a body comprising an upper housing, a lower housing, a bottom, skin-contacting base, and an infusion tubing extending outward from the body configured to connect to a source of the therapeutic fluid; a sensing cannula comprising a proximal end, a distal end, an external surface, an internal lumen, at least one hollow channel within the internal lumen extending from the proximal end of the sensing cannula to the distal end of the sensing cannula configured for the administration of the therapeutic fluid, at least one indicating electrode on the external surface configured to sense the concentration of the analyte, and a conductor on the external surface extending from the proximal end of the sensing cannula to the at least one indicating electrode, wherein the proximal end of the sensing cannula is retained within the body, and wherein the distal end of the sensing cannula extends from the skin-contacting base; a channel within the body in fluid communication with the internal cavity formed by the self-sealing septum and the proximal end of the combined sensing cannula; a signal processing module, comprising a second body comprising an upper face, a lower face, and a vertical surface between the upper face and the lower face, wherein the vertical surface provides an electrical potential to the sensing cannula and receives an electrical current from the sensing cannula via a set of electrical contacts on the vertical surface, wherein the second body comprises a set of arms in contact with the upper housing, and wherein the lower face is in contact with the skin-contacting base; and an interface circuit comprising a proximal end and a distal end, wherein the interface circuit comprises one or more conductors configured to convey current signals from the sensing cannula to the signal processing module, wherein the proximal end of the interface circuit is in electrical contact with the proximal end of the sensing cannula, and wherein the distal end of the interface circuit is in electrical contact with the signal processing module.

In some embodiments, the infusion tubing is reversibly attached to the body a connector comprising one or more cantilever snap joints configured to permit the reversible attachment of the infusion tubing. In some embodiments, the at least one indicating electrode comprises an enzyme layer overlaying a conductive surface. In some embodiments, the enzyme layer is covered with a semi-permeable membrane. In some embodiments, the enzyme layer comprises glucose oxidase or glucose dehydrogenase. In some embodiments, the enzyme layer includes an osmium-based redox mediator. In some embodiments, the osmium-based redox mediator comprises osmium dimethyl bipyridine. In some embodiments, the enzyme layer comprises polyvinylimidazole. In some embodiments, the sensing cannula comprises a reference electrode comprising silver/silver chloride (Ag/AgCl). In some embodiments, the signal processing module provides a bias potential to the sensing cannula of less than 250 millivolts (mV) relative to a reference potential. In some embodiments, the channel comprises a stainless steel needle connecting from the cavity to the proximal end of the sensing cannula. In some embodiments, the upper housing and the lower housing are configured to receive a hollow inserter needle partially enclosing the sensing cannula for insertion into a skin surface of a mammal. In some embodiments, the sensing cannula comprises a stiffness sufficient for insertion into a skin surface of a mammal without using an inserter needle. In some embodiments, the skin-contacting base comprises an adhesive surface configured to attach the device to a skin surface of a subject. In some embodiments, the analyte is selected from the group consisting of: oxygen, glucose, lactate, a drug metabolite, and a pathogen. In some embodiments, the analyte is glucose. In some embodiments, the therapeutic fluid is selected from the group consisting of: an insulin or insulin analog formulation, glatiramer acetate, heparin, human menopausal gonadotropin, vitamins, and minerals. In some embodiments, the therapeutic fluid is the insulin or the insulin analog formulation. In some embodiments, the insulin or the insulin analog formulation comprises an excipient comprising a phenol or cresol.

In another aspect, the present disclosure provides a device configured to perform simultaneous sensing of a concentration of an analyte and administration of a therapeutic fluid, comprising: a body comprising an upper housing, a lower housing, and a bottom, skin-contacting base, wherein the upper housing comprises a port configured to reversibly attach to a fluid delivery device configured for delivery of a fluid via insertion of a needle, wherein the port comprises a self-sealing septum in contact with the lower housing thereby forming an internal cavity; a sensing cannula comprising a proximal end, a distal end, an external surface, an internal lumen, at least one hollow channel within the internal lumen extending from the proximal end of the sensing cannula to the distal end of the sensing cannula configured for the administration of the therapeutic fluid, at least one indicating electrode on the external surface configured to sense the concentration of the analyte, and a conductor on the external surface extending from the proximal end of the sensing cannula to the at least one indicating electrode, wherein the proximal end of the sensing cannula is retained within the body, and wherein the distal end of the sensing cannula extends from the skin-contacting base; and a channel within the body in fluid communication with the internal cavity formed by the self-sealing septum and the proximal end of the combined sensing cannula.

In some embodiments, the upper housing comprises a top face comprising the port. In some embodiments, the port comprises a visible opening comprising the self-sealing septum. In some embodiments, the device further comprises a signal processing module configured to receive an electrical current from the sensing cannula. In some embodiments, the signal processing module is configured to provide an electrical potential to the sensing cannula. In some embodiments, the signal processing module comprises a second body comprising an upper face, a lower face, and a vertical surface between the upper face and the lower face. In some embodiments, the vertical surface provides the electrical potential to the sensing cannula and receives the electrical current from the sensing cannula via a set of electrical contacts on the vertical surface. In some embodiments, the second body comprises a set of arms in contact with the upper housing, and wherein the lower face is in contact with the skin-contacting base. In some embodiments, the device further comprises an interface circuit configured to convey current signals from the sensing cannula to the signal processing module. In some embodiments, the interface circuit comprises a proximal end and a distal end. In some embodiments, the interface circuit comprises one or more conductors configured to convey the current signals from the sensing cannula to the signal processing module. In some embodiments, the proximal end of the interface circuit is in electrical contact with the proximal end of the sensing cannula, and wherein the distal end of the interface circuit is in electrical contact with the signal processing module. In some embodiments, the fluid delivery device comprises a syringe or a pen. In some embodiments, the fluid delivery device comprises a syringe. In some embodiments, the fluid delivery device comprises a pen. In some embodiments, the at least one indicating electrode comprises an enzyme layer overlaying a conductive surface. In some embodiments, the enzyme layer is covered with a semi-permeable membrane. In some embodiments, the enzyme layer comprises glucose oxidase or glucose dehydrogenase. In some embodiments, the enzyme layer comprises an osmium-based redox mediator. In some embodiments, the osmium-based redox mediator comprises osmium dimethyl bipyridine. In some embodiments, the enzyme layer comprises polyvinylimidazole. In some embodiments, the sensing cannula comprises a reference electrode comprising silver/silver chloride (Ag/AgCl). In some embodiments, the signal processing module provides a bias potential to the sensing cannula of less than 250 millivolts (mV) relative to a reference potential. In some embodiments, the channel comprises a stainless steel needle connecting from the cavity to the proximal end of the sensing cannula. In some embodiments, the upper housing and the lower housing are configured to receive a hollow inserter needle partially enclosing the sensing cannula for insertion into a skin surface of a mammal. In some embodiments, the sensing cannula comprises a stiffness sufficient for insertion into a skin surface of a mammal without using an inserter needle. In some embodiments, the skin-contacting base comprises an adhesive surface configured to attach the device to a skin surface of a subject. In some embodiments, the analyte is selected from the group consisting of: oxygen, glucose, lactate, a drug metabolite, and a pathogen. In some embodiments, the analyte is glucose. In some embodiments, the therapeutic fluid is selected from the group consisting of: an insulin or insulin analog formulation, glatiramer acetate, heparin, human menopausal gonadotropin, vitamins, and minerals. In some embodiments, the therapeutic fluid is the insulin or the insulin analog formulation. In some embodiments, the insulin or the insulin analog formulation comprises an excipient comprising a phenol or cresol.

In another aspect, the present disclosure provides A device configured to perform simultaneous sensing of a concentration of an analyte and administration of a therapeutic fluid, comprising: a body comprising an upper housing, a lower housing, a bottom, skin-contacting base, and an infusion tubing extending outward from the body configured to connect to a source of the therapeutic fluid; a sensing cannula comprising a proximal end, a distal end, an external surface, an internal lumen, at least one hollow channel within the internal lumen extending from the proximal end of the sensing cannula to the distal end of the sensing cannula configured for the administration of the therapeutic fluid, at least one indicating electrode on the external surface configured to sense the concentration of the analyte, and a conductor on the external surface extending from the proximal end of the sensing cannula to the at least one indicating electrode, wherein the proximal end of the sensing cannula is retained within the body, and wherein the distal end of the sensing cannula extends from the skin-contacting base; and a channel within the body in fluid communication with the internal cavity formed by the self-sealing septum and the proximal end of the combined sensing cannula.

In some embodiments, the device further comprises a signal processing module configured to receive an electrical current from the sensing cannula. In some embodiments, the signal processing module is configured to provide an electrical potential to the sensing cannula. In some embodiments, the signal processing module comprises a second body comprising an upper face, a lower face, and a vertical surface between the upper face and the lower face. In some embodiments, the vertical surface provides the electrical potential to the sensing cannula and receives the electrical current from the sensing cannula via a set of electrical contacts on the vertical surface. In some embodiments, the second body comprises a set of arms in contact with the upper housing, and wherein the lower face is in contact with the skin-contacting base. In some embodiments, the device further comprises an interface circuit configured to convey current signals from the sensing cannula to the signal processing module. In some embodiments, the interface circuit comprises a proximal end and a distal end. In some embodiments, the interface circuit comprises one or more conductors configured to convey the current signals from the sensing cannula to the signal processing module. In some embodiments, the proximal end of the interface circuit is in electrical contact with the proximal end of the sensing cannula, and wherein the distal end of the interface circuit is in electrical contact with the signal processing module. In some embodiments, the infusion tubing is reversibly attached to the body a connector comprising one or more cantilever snap joints configured to permit the reversible attachment of the infusion tubing. In some embodiments, the at least one indicating electrode comprises an enzyme layer overlaying a conductive surface. In some embodiments, the enzyme layer is covered with a semi-permeable membrane. In some embodiments, the enzyme layer comprises glucose oxidase or glucose dehydrogenase. In some embodiments, the enzyme layer includes an osmium-based redox mediator. In some embodiments, the osmium-based redox mediator comprises osmium dimethyl bipyridine. In some embodiments, the enzyme layer comprises polyvinylimidazole. In some embodiments, the sensing cannula comprises a reference electrode comprising silver/silver chloride (Ag/AgCl). In some embodiments, the signal processing module provides a bias potential to the sensing cannula of less than 250 millivolts (mV) relative to a reference potential. In some embodiments, the channel comprises a stainless steel needle connecting from the cavity to the proximal end of the sensing cannula. In some embodiments, the upper housing and the lower housing are configured to receive a hollow inserter needle partially enclosing the sensing cannula for insertion into a skin surface of a mammal. In some embodiments, the sensing cannula comprises a stiffness sufficient for insertion into a skin surface of a mammal without using an inserter needle. In some embodiments, the skin-contacting base comprises an adhesive surface configured to attach the device to a skin surface of a subject. In some embodiments, the analyte is selected from the group consisting of: oxygen, glucose, lactate, a drug metabolite, and a pathogen. In some embodiments, the analyte is glucose. In some embodiments, the therapeutic fluid is selected from the group consisting of: an insulin or insulin analog formulation, glatiramer acetate, heparin, human menopausal gonadotropin, vitamins, and minerals. In some embodiments, the therapeutic fluid is the insulin or the insulin analog formulation. In some embodiments, the insulin or the insulin analog formulation comprises an excipient comprising a phenol or cresol.

In some embodiments, the body is circular or substantially circular, with an accessible surface on one face having a self-sealing inlet port; a skin contact surface on the opposite face, with a combined sensor and cannula projecting outward therefrom; a liquid delivery channel connecting the inlet port to the cannula; a cavity that accepts an electronic signal processing device; a retention mechanism for the signal processing device; and an electrical contact between the signal processing device and the sensor.

In some embodiments, the body is round or oval, or substantially round or oval, with an accessible surface on one face having a self-sealing inlet port; a skin contact surface on the opposite face, with a combined sensor and cannula projecting outward therefrom; a liquid delivery channel connecting the inlet port to the cannula; an electronic signal processing device with a set of arms that attach it to the housing of the liquid delivery channel; a retention mechanism for the signal processing device; and an electrical contact between the signal processing device and the sensor.

In some embodiments, the body is oval or substantially oval, with an accessible surface on one face having a self-sealing inlet port; a skin contact surface on the opposite face, with a combined sensor and cannula projecting outward therefrom; a liquid delivery channel connecting the inlet port to the cannula; an electronic signal processing device that attaches to a vertical face of said body; a retention mechanism for the signal processing device; and an electrical contact between the signal processing device and the sensor.

In some embodiments, the body is circular or oval, or substantially circular or oval, with an accessible surface on one face having a segment of infusion tubing projecting therefrom; a skin contact surface on the opposite face, with a combined sensor and cannula projecting outward therefrom; a liquid delivery tube connecting said infusion tubing to the cannula; a set of retention arms designed to align and retain the electronic signal processing device; features designed to receive the attachment arms of the electronic signal processing device; and an electrical contact interface between the signal processing device and the sensor.

In some embodiments, the body is essentially circular or oval, with an accessible surface on one face having a segment of infusion tubing projecting therefrom; a skin contact surface on the opposite face, with a combined sensor and cannula projecting outward therefrom; a liquid delivery tube connecting said infusion tubing to said cannula; a self-sealing port connected to said liquid delivery channel; retention arms designed to align and retain and electronic signal processing device; features designed to receive the attachment arms of said electronic signal processing device; and an electrical contact interface between said signal processing device and said sensor.

In some embodiments, the cannula projects outward from the skin contact surface at an angle between 40 and 60 degrees. In some embodiments, the cannula projects outward from the skin contact surface perpendicularly or substantially perpendicularly.

In some embodiments, the device is configured to be inserted or driven into the skin using an insertion device. The insertion device may make temporary contact with the accessible surface of the body. In some embodiments, the cannula has a fluid path that is composed essentially of a flexible polymer and is placed in the tissue using a rigid inserter element or trocar that is removed immediately following insertion. In some embodiments, the insertion device comprises an insertion needle piercing the self-sealing inlet port, passing through the liquid delivery channel, and extending just beyond the distal end of the cannula. In some embodiments, the cannula comprises a fluid path formed by a permanently fixed needle that can be placed in the tissue and remains for the duration of use.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

Embodiments will be readily understood by the following detailed description in conjunction with the accompanying drawings and the appended claims. Embodiments are illustrated by way of example and not by way of limitation in the figures of the accompanying drawings.

FIG. 1A provides a perspective view of an example of a combined CGM infusion port with an internal removable electronic module.

FIG. 1B provides another perspective view of the combined CGM infusion port of FIG. 1A, with the internal removable electronic module removed.

FIG. 2 provides an exploded view of the combined CGM infusion port of FIG. 1A.

FIGS. 3A-3C provide sectional views of an example of a combined CGM infusion port with an internal removable electronic module and an insertion device.

FIG. 4 provides a cross-sectional view of an example of a combined CGM infusion port with an internal removable electronic module.

FIG. 5 provides a cross-sectional view of an example of a combined CGM infusion port with an internal removable electronic module, with the fluid delivery device inserted into the skin of a subject (e.g., a patient), with a syringe positioned within the device to provide fluid delivery (e.g., drug delivery) to the subject.

FIGS. 6A-6B provide perspective views of an example of a combined CGM infusion port with an external removable electronic module.

FIGS. 7A-7B provide exploded views of the combined CGM infusion port of FIGS. 6A-6B, including a view of an inserter needle (FIG. 7B).

FIGS. 8A-8D provide cross-sectional views of an example of a combined CGM infusion port, including views of the interconnect detail. FIG. 8A shows a side section view, FIG. 8B shows a front section view, FIG. 8C shows a side section view showing a fluid path and electrical contact detail, and FIG. 8D shows a front section view showing a fluid path and electrical contact detail.

FIGS. 9A-9D provide cross-sectional views of an example of a combined CGM infusion port in contact with a needle-free insulin pen tip. FIG. 9A shows a side section view, FIG. 9B shows a front section view, FIG. 9C shows a side section view showing a fluid path and electrical contact detail, and FIG. 9D shows a front section view showing a fluid path and electrical contact detail.

FIGS. 10A-10G provide views of an example of a disposable CGM infusion port in contact with a pen having a needle-free insulin pen tip. FIG. 10A provides a perspective view of the disposable CGM infusion port with the pen tip attached. FIG. 10B provides a perspective view of the disposable CGM infusion port including the internal structure (e.g., electronics).

FIG. 10C provides a cutaway view of the disposable CGM infusion port with the pen tip attached, including the fluid path. FIG. 10D provides a sectional view of the disposable CGM infusion port including the sensor electrical interconnect detail. FIGS. 10E-10G provide sectional views of the disposable CGM infusion port in contact with a pen having a needle-free insulin pen tip, including a sectional view with the pen tip attached (FIG. 10E), detail of the fluid path section with the pen tip disengaged from the fluid path (FIG. 10F), and details of the fluid path section with the pen tip engaged with the fluid path (FIG. 10G).

FIGS. 11A-11B provide views of an example of a combined CGM infusion port with a rigid sensor, including a front section view (FIG. 11A) and a front section view showing a fluid path and electrical contact detail (FIG. 11B).

FIGS. 12A-12C provide perspective views (FIGS. 12A-12B) and an exploded view (FIG. 12C) of an example of a combined CGM infusion port configured for attachment to an insulin pump or gravity-fed source of medication.

FIGS. 13A-13B provide a perspective view (FIG. 13A) and a top sectional view (FIG. 13B) of an example of a combined CGM infusion port configured for attachment to an insulin pump or gravity-fed source of medication with electronic module removed, showing a fluid path and electrical interconnect detail.

FIGS. 14A-14D provide a perspective view (FIG. 14A), a top sectional view (FIG. 14B), a front sectional view (FIG. 14C), and a side sectional view (FIG. 14D) of an example of a combined CGM infusion port configured for attachment to an insulin pump or gravity-fed source of medication with a rigid inserter needle or trocar. FIGS. 14-14B show the interconnect to electronics. FIGS. 14-14D show the tubed infusion set.

### DETAILED DESCRIPTION

References are made herein to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

As used herein, the term “cannula” generally refers to a hollow tube fabricated using a rigid material, such as a polymer or a metal, having an interior (e.g., inner) surface and an exterior (e.g., outer) surface, and an opening at both ends.

As used herein, the term “sensing cannula” generally refers to a cannula having an analyte sensor disposed on the exterior surface and one or more fluid delivery channels contained within the cannula.

As used herein, the term “continuous glucose monitor (CGM)” generally refers to a system comprising electronics configured for continuous or nearly continuous measurement of glucose levels from a subject (e.g., a human being, an animal, or a mammal) and/or reporting of such measurements.

As used herein, the term “CGM injection port” generally refers to a device (e.g., a unified device) configured for use on the skin of a subject (e.g., a human being, an animal, or a mammal) having a combination of a sensor and a cannula that includes an electrical interface to signal acquisition electronics and a port for attachment of a fluid source such as an insulin pen, a syringe, or another fluid delivery device.

As used herein, the term “CGM infusion set” generally refers to a device (e.g., a unified device) configured for use on the skin of a subject (e.g., a human being, an animal, or a mammal) having a combination of a sensor and a cannula that includes an electrical interface to signal acquisition electronics and a port for attachment of a fluid source such as a pump or a gravity-fed sourced source.

The terms “coupled” and “connected,” along with their derivatives, may be used herein. It should be understood that these terms are not intended as synonyms for each other. Rather, in particular embodiments, “connected” may be used to indicate that two or more elements are in direct physical or electrical contact with each other. “Coupled” may be used to indicate that two or more elements are in direct physical or electrical contact. However, “coupled” may also be used to indicate that two or more elements are not in direct contact with each other, but yet still cooperate or interact with each other.

As used herein, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B, and C). For the purposes of the description, a phrase in the form “(A)B” means (B) or (AB) that is, A is an optional element.

As used herein, the terms “embodiment” or “embodiments,” may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, the plural can be translated to the singular and/or the singular can be translated to the plural, as is appropriate to the context and/or application. The various singular/plural permutations may be expressly set forth herein for sake of clarity.

There are a growing number of medical therapies that involve the subcutaneous infusion of liquid treatments. For example, glatiramer acetate, a treatment for multiple sclerosis, may be prescribed for daily subcutaneous injection. As another example, heparin may be administered via frequent subcutaneous injection as a treatment for certain clotting disorders. As another example, human menopausal gonadotropin is injected subcutaneously on a daily basis in women underdoing fertility treatments. As another example, pediatric patients undergoing parenteral nutrition supplementation may receive repeated subcutaneous doses of multivitamins. Subcutaneous injections are also commonly used in veterinary applications.

One of the largest populations on daily subcutaneous injections is individuals with insulin-treated Type 1 or Type 2 diabetes mellitus. Most such subjects may administer more than one injection per day, a regimen known as multiple daily injection (MDI) therapy. For example, an infusion port for medication delivery may be designed to be attached to the skin surface, with a percutaneous cannula that extends perpendicularly from the base (e.g., as described in U.S. Pat. No. 7,338,465, which is incorporated by reference herein in its entirety). Following insertion with an insertion needle, the cannula remains in the subcutaneous tissue over multiple days to deliver medication without the need for additional painful injections.

Amperometric analyte sensors may be used to detect various analytes as oxygen, pH, glucose, lactate, drug metabolites, and pathogens in vivo. Further, sensors for Continuous Glucose Monitoring (CGM) may have widespread clinical adoption. These CGM sensors may reside in the subcutaneous tissue, and generate small glucose-dependent electrical currents that are detected by associated electronics.

In many instances, it is desirable to both track the concentration of an analyte and deliver a medication in response to the level of the analyte. For example, this may be performed in the case of glucose analyte monitoring and insulin medication delivery, as insulin pumps may feature automated insulin dosing based upon readings from a CGM sensor. For the convenience of the user, it may be desirable to combine both sensing and infusion into a single device. However, despite the availability of both CGM sensors and infusion ports, there remain challenges in realizing a single unified device that effectively combines the two functions. Consequently, automated insulin dosing pumps may use physically separated sensors and infusion sites. This multiplicity of sites requires additional time to manage, increases pain and infection risk, and increases cost to the patient.

In the specific case of glucose measurement, integration may be prevented by, among other things, an assumption that insulin delivery in proximity to a glucose sensor in diabetes management of a patient necessarily corrupts sensor readings due to local uptake of the analyte. Therefore, many commercially available CGM devices use a separation distance between the site of insulin delivery and glucose monitoring. For example, Dexcom's G6 instructions instruct the user to “choose a site at least 3 inches from insulin pump infusion set or injection site” (p. 11 of Dexcom G6 User Guide, 2017, which is incorporated by reference herein in its entirety). Likewise, Abbott instructions instruct the user to keep its Libre sensor “at least 1 inch away from an insulin injection site” (p. 21 of Libre In-Service Guide, Abbott ADC-05821 v2.0, October 2017, which is incorporated by reference herein in its entirety). Further, Medtronic advises the user to use the CGM sensor “1 inch from your insulin pump infusion site” and “1 inch from any manual insulin injection site.” (p. 12 of My Guardian Connect manual, Medtronic, Apr. 27, 2018, which is incorporated by reference herein in its entirety).

Using current devices, every insertion site for insulin injection may require piercing the skin with a separate needle that may be painful for the patient, and each insertion site may bring with it the risk of complications such as scarring and infection. The physical separation and resulting complexity also increases the cost and size of the device worn on the body. In order to be less painful and more convenient and discreet for the patient, as well as less expensive, the present disclosure provides improved devices, systems, and methods for a unified analyte sensing fluid delivery cannula. Such improved devices, systems, and methods feature a glucose sensor that is directly disposed on the surface of the infusion cannula. The physiological effect of insulin on surrounding subcutaneous tissue glucose concentration has been demonstrated to be negligible, since it has been discovered that the greater effect on amperometric glucose sensors is in fact interference from electroactive components of the insulin excipient that cause the sensor current to initially rise, followed by a permanent loss of glucose sensitivity. Therefore, it is possible to measure interstitial fluid glucose levels in the immediate vicinity of insulin delivery through the use of an appropriately designed amperometric glucose sensor (e.g., as described in US Pat. Pub. No. US 2016/0354542 A1, which is incorporated by reference herein in its entirety).

In light of the challenges outlined above, the present disclosure provides infusion devices to satisfy the need for reliable and viable solutions for the attachment of a unified sensing cannula to necessary signal processing electronics and common fluid infusion devices. Such infusion devices may enable the simultaneous connection of an amperometric sensor on the surface of an infusion cannula to signal processing electronics and various suitable drug delivery mechanisms, including syringes, pens, and pumps to the fluid path of the same infusion cannula.

The present disclosure provides systems and devices for combining analyte monitoring with fluid delivery, including devices that are adapted for use with combined sensors and cannulas having sensors and cannulas on a single component. These systems and devices may be used in applications with in vivo monitoring of analyte concentrations (e.g., pH, oxygen, lactate, glucose, and insulin concentration) and delivery of medications (e.g., glatiramer acetate, heparin, human menopausal gonadotropin, insulin, and vitamin and nutrient supplements). These systems and devices may be used in various applications in various situations, such as treatment of multiple sclerosis, fertility treatments, diabetes, nutritional supplementation, and automated drug dosing.

Infusion devices of the present disclosure may be configured to be attached to the skin surface of a subject (e.g., patient), with a single combined sensing cannula penetrating the skin surface into the subcutaneous compartment of the subject. These devices may be configured for use with an external fluid source, such as an insulin syringe, insulin pen, smart pen, or infusion pump. Once properly inserted on the body, the device can be used to deliver fluid to the patient for a prolonged period of time (e.g., 3 days or more), thereby avoiding the pain and inconvenience of several needle sticks in that time frame.

Infusion devices of the present disclosure may also have the advantage of a smaller size than other infusion devices that include an amperometric sensor. Infusion devices of the present disclosure, instead of requiring two separate devices on the body, may have only a single component attached to or penetrating into the skin. As compared to other devices for analyte sensing and drug delivery in a common assembly, the physical separation required of this approach may set physical or practical limitations (e.g., a lower bound) on the size of the device, which are relieved by systems and devices of the present disclosure. Further, other devices for analyte sensing and drug delivery in a common assembly may fail to sufficiently integrate an electronic interface, which may add non-trivial and considerable additional size and complexity to a functional solution. Significant challenges may be presented or associated with co-location of electrical and fluid-handling features on a single percutaneous device, as both the electrical and fluid interfaces may need to be accomplished in a limited space. Further, the ability of the sensor to record signal currents accurately may be compromised by reliability issues, such as a leakage of fluid into the electrical interface. Systems and devices of the present disclosure provide a sensor and a fluid delivery cannula that is capable of handling electrical and fluid path connections thereto.

Recognizing the need for improved combined CGM infusion port devices that avoid the use of multiple insertion needles, systems and devices of the present disclosure combine a sensor and cannula with an insertion system that can place or insert the unified sensing cannula into a subject (e.g., a patient) without damaging the fluid and electrical connections. Further, systems and devices of the present disclosure provide suitable solutions for insertion while meeting constraints on the fluid and electrical connections themselves.

In various embodiments, systems and devices of the present disclosure effectively provide solutions for electronic processing of sensor signals via an electronic signal processing module, which is configured to facilitate the electromechanical interface between the sensor contacts and signal processing hardware. These enable the temporary or permanent electrical connection between sensor and associated processing electronics, and permit the reuse of the electronics if desired.

In some embodiments, the body is circular or substantially circular, with an accessible surface on one face having a self-sealing inlet port; a skin contact surface on the opposite face, with a combined sensor and cannula projecting outward therefrom; a liquid delivery channel connecting the inlet port to the cannula; a cavity that accepts an electronic signal processing device; a retention mechanism for the signal processing device; and an electrical contact between the signal processing device and the sensor.

In some embodiments, the body is round or oval, or substantially round or oval, with an accessible surface on one face having a self-sealing inlet port; a skin contact surface on the opposite face, with a combined sensor and cannula projecting outward therefrom; a liquid delivery channel connecting the inlet port to the cannula; an electronic signal processing device with a set of arms that attach it to the housing of the liquid delivery channel; a retention mechanism for the signal processing device; and an electrical contact between the signal processing device and the sensor.

In some embodiments, the body is oval or substantially oval, with an accessible surface on one face having a self-sealing inlet port; a skin contact surface on the opposite face, with a combined sensor and cannula projecting outward therefrom; a liquid delivery channel connecting the inlet port to the cannula; an electronic signal processing device that attaches to a vertical face of said body; a retention mechanism for the signal processing device; and an electrical contact between the signal processing device and the sensor.

In some embodiments, the body is circular or oval, or substantially circular or oval, with an accessible surface on one face having a segment of infusion tubing projecting therefrom; a skin contact surface on the opposite face, with a combined sensor and cannula projecting outward therefrom; a liquid delivery tube connecting said infusion tubing to the cannula; a set of retention arms designed to align and retain the electronic signal processing device; features designed to receive the attachment arms of the electronic signal processing device; and an electrical contact interface between the signal processing device and the sensor.

In some embodiments, the body is essentially circular or oval, with an accessible surface on one face having a segment of infusion tubing projecting therefrom; a skin contact surface on the opposite face, with a combined sensor and cannula projecting outward therefrom; a liquid delivery tube connecting said infusion tubing to said cannula; a self-sealing port connected to said liquid delivery channel; retention arms designed to align and retain and electronic signal processing device; features designed to receive the attachment arms of said electronic signal processing device; and an electrical contact interface between said signal processing device and said sensor.

In some embodiments, the cannula projects outward from the skin contact surface at an angle between 40 and 60 degrees. In some embodiments, the cannula projects outward from the skin contact surface perpendicularly or substantially perpendicularly.

In some embodiments, the device is configured to be inserted or driven into the skin using an insertion device. The insertion device may make temporary contact with the accessible surface of the body. In some embodiments, the cannula has a fluid path that is composed essentially of a flexible polymer and is placed in the tissue using a rigid inserter element or trocar that is removed immediately following insertion. In some embodiments, the insertion device comprises an insertion needle piercing the self-sealing inlet port, passing through the liquid delivery channel, and extending just beyond the distal end of the cannula. In some embodiments, the cannula comprises a fluid path formed by a permanently fixed needle that can be placed in the tissue and remains for the duration of use.

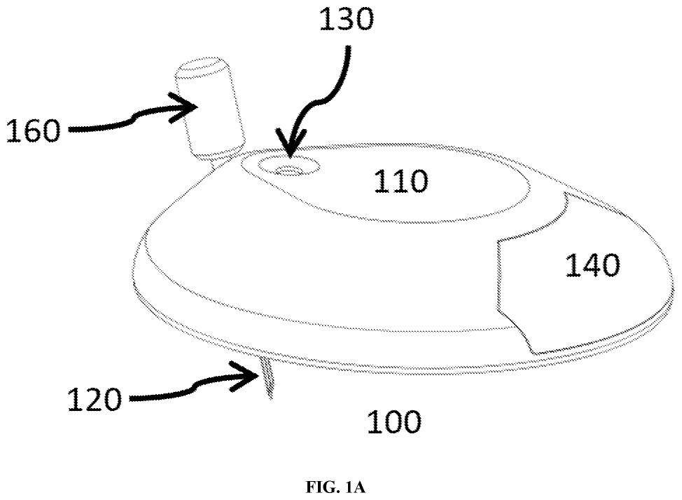

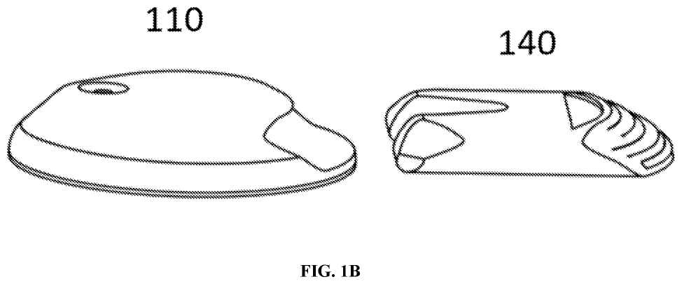

FIGS. 1A-1B provide perspective views of an example of a combined CGM infusion port 100 with an internal removable electronic module. The combined CGM infusion port 100 includes a body 110, a sensing cannula 120 projecting downward from the body, an access port 130 on the top surface of the body, and an electronic signal processing module 140 enclosed within the body. An adhesive patch 116 provides for adhesive attachment to a subject (e.g., a patient). An access port 130 permits a user (e.g., a subject, a patient, a physician, a nurse, a clinician, or a caretaker of the subject) to attach a fluid delivery device (e.g., a syringe, pen needle, or insulin pump) to the subject. This fluid may be a drug, diagnostic agent, or other liquid that is desired for subcutaneous infusion. An inserter 160 allows the user to insert the cannula into the skin of the subject.

As shown in FIG. 1B, in some embodiments, the electronic signal processing module 140 may be removable and is shown separated from infusion device body 110. The infusion components, such as the cannula, may be disposable and have a use life limited to 3 or more days. By configuring the electronic signal processing module such that it may be removed, it may be reused repeatedly, thereby reducing the recurring cost of the system. However, in other embodiments, the transmitter is permanently affixed inside the infusion device body and is discarded with the infusion device.

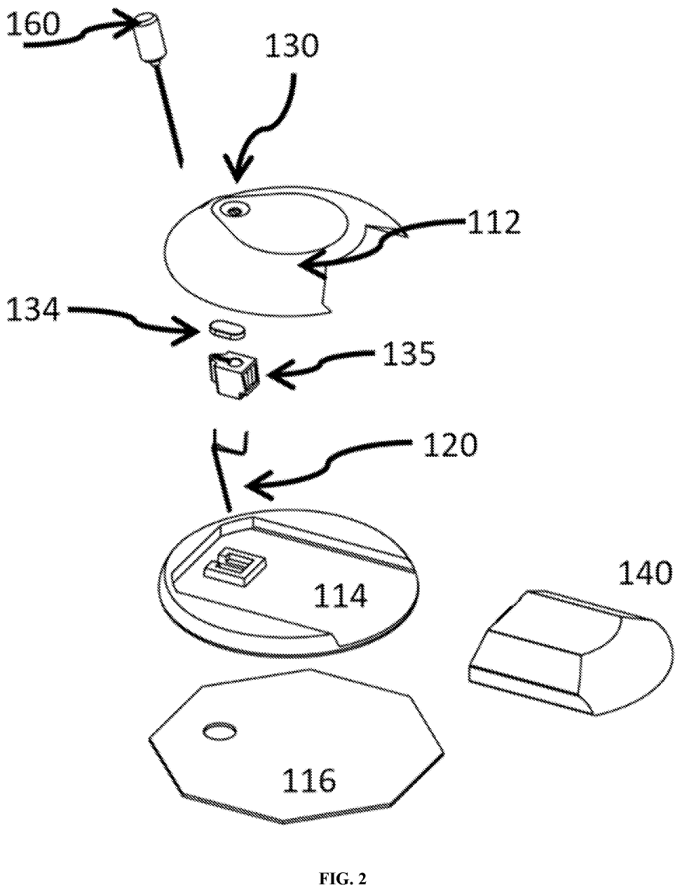

FIG. 2 provides an exploded view of the combined CGM infusion port of FIG. 1A. The body 110 is shown separated into an upper housing 112 and a base 114, and the sensing cannula 120 is separated from the base 114. These pieces may comprise a material such as injection molded plastic and be bonded to one another via adhesive, ultrasonic welding, or other techniques for joining of plastics. The adhesive patch 116 provides for attachment to the subject (e.g., patient) on the bottom face, and adhesive attachment to the base 114 on its top face. The sensing cannula 120 and access port 130 are shown prior to assembly. A self-sealing septum 134 and fluid path housing 135 serve to provide intermittent connection between a fluid delivery device and the fluid path of cannula 120. The electronic signal processing module 140 is shown removed from the body.

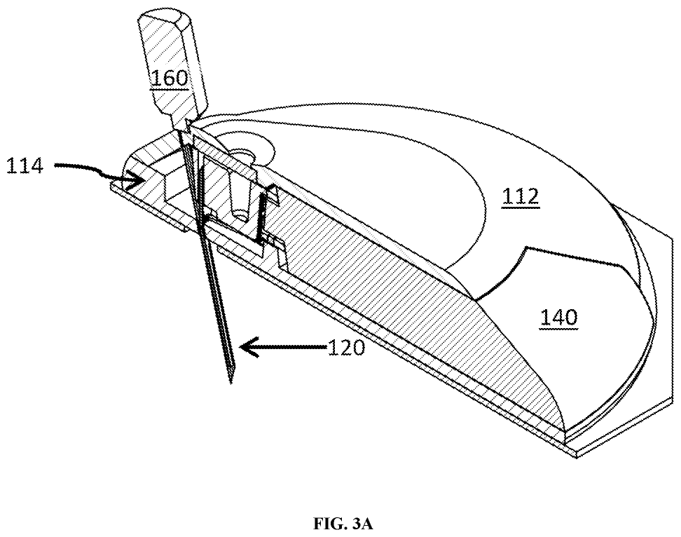

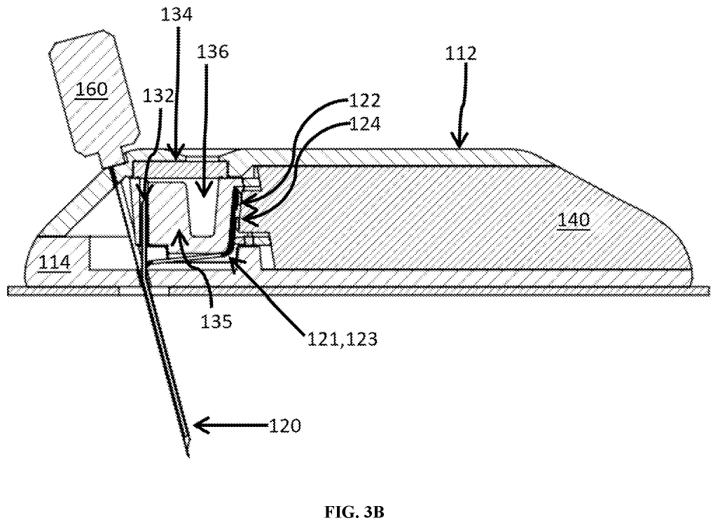

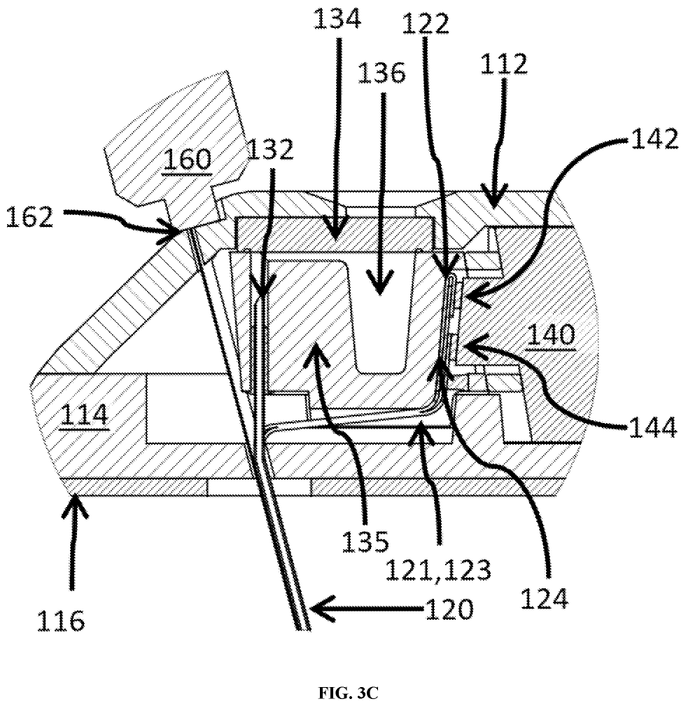

FIGS. 3A-3C provide sectional views of an example of a combined CGM infusion port with an internal removable electronic module and an insertion device used to place the cannula into the subcutaneous tissue. In this configuration, the sensing cannula has sufficiently long conductors to make direct contact with the electronic module. An opening 162 allows passage of an inserter 160 through the upper housing 112. The inserter cross section is hollow and may be round or roughly square (e.g., having three sides with the fourth side open). An opening in the cross section permits the fluid path connection, formed by a tube 132 extending out of the sensing cannula 120, to pass outside of the hollow inserter and make fluid connection with a needle cavity 136 formed by the fluid path body 135. Fluid is delivered into the subcutaneous tissue of the subject by inserting a needle through a septum 134 to access the needle cavity 136. The opening in the inserter also permits the passage of the sensor conductors 121 and 123, which are in electrical communication with a set of contacts 122 and 124 at the proximal end of the sensing cannula 120. The set of contacts 122 and 124 make physical and electrical contact with a set of sensor electronic module contacts 142 and 144 on the electronic signal processing module 140.

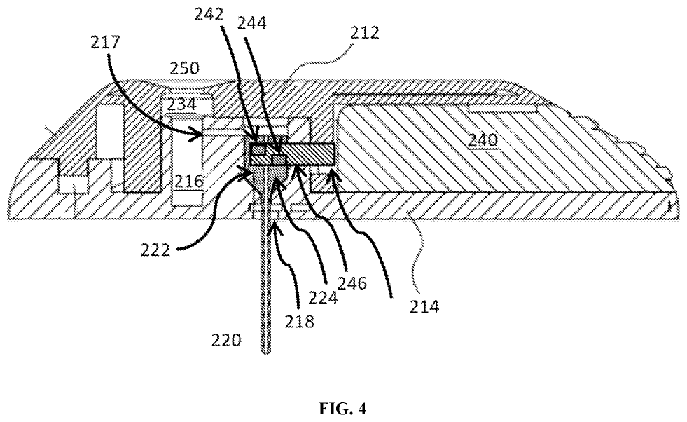

FIG. 4 provides a cross-sectional view of an example of a combined CGM infusion port with an internal removable electronic module. The device features co-located electrical connections for analyte sensing and fluid delivery on a unified analyte sensing cannula, which is configured for use with an intermittently connected fluid source (e.g., a syringe or a pen). The electronic signal processing module 240 is shown inserted in the cavity formed by body 210. An electrical connection to the sensing cannula 220 from the electronic signal processing module 240 is provided via a flexible electrical connector 246 making electrical contact with electronic signal processing module 240 via a set of contacts 242 and 244, and held in contact with contacts 222 and 224 at the proximal end of the combined analyte sensor and infusion cannula 220. A fluid connection to proximal end of the combined analyte sensor and infusion cannula 220 is provided via an opening 219 in the base 214 that permits fluid to flow from the adjacent needle cavity 216 into the infusion cannula. The sensing cannula 220 exits through the base 214 through an opening 218. Access to the needle cavity 216 is provided through an opening 250 in the upper housing 212 and through penetration of the self-sealing septum 234 by the fluid delivery device. Fluid flows from needle cavity 216 to the sensing cannula 220 via a channel 217. In this embodiment, the sensing cannula 220 may be placed into the skin of a subject with the aid of an insertion device, or it may be capable of piercing the skin of the subject without the need for a temporary inserter needle.

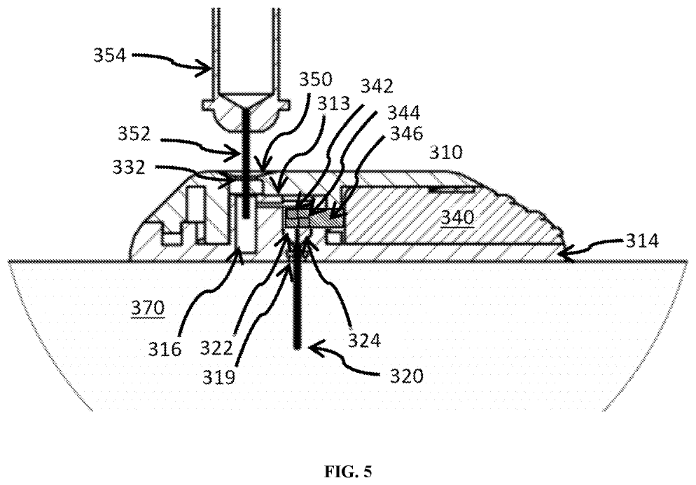

FIG. 5 provides a cross-sectional view of an example of a combined CGM infusion port with an internal removable electronic module, with the fluid delivery device inserted into the skin of a subject (e.g., a patient), with a syringe positioned within the device to provide fluid delivery (e.g., drug delivery) to the subject in an example application. The unified sensing cannula 320 is embedded in the subcutaneous tissue 370, essentially perpendicular to the plane of the skin surface. A fluid delivery device 354 is shown with a needle 352 inserted through an opening 350 and a self-sealing septum 332 into a cavity 316. The fluid delivery device may be selected from various suitable fluid sources, such as a syringe, an insulin pen, a drug infusion pump, and a gravity-fed fluid source. An electronic signal processing module 340 is shown inserted in the cavity 316 formed by the body 310. An electrical connection to the sensing cannula 320 from the electronic signal processing module is provided via a flexible electrical circuit 346, having a set of electrical contacts 342 and 344, held in contact with a set of contacts 322 and 324 at the proximal end of the sensing cannula 320. A permanent, waterproof connection is provided from the set of sensor contacts 322 and 324 to the set of flex circuit contacts 342 and 344 by a waterproof, conductive adhesive, and may be further encapsulated in a non-conductive waterproof barrier, such as an epoxy-based encapsulant. A fluid connection to proximal end of the combined analyte sensor and infusion cannula 320 is provided via an opening 313 in the base 314 that permits fluid to flow from the adjacent needle cavity 316. The sensing cannula 320 exits through the base 314 through an opening 319.

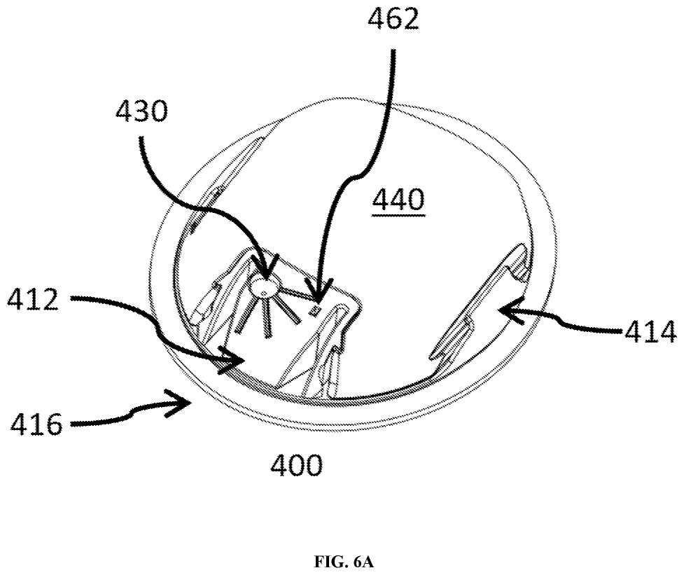

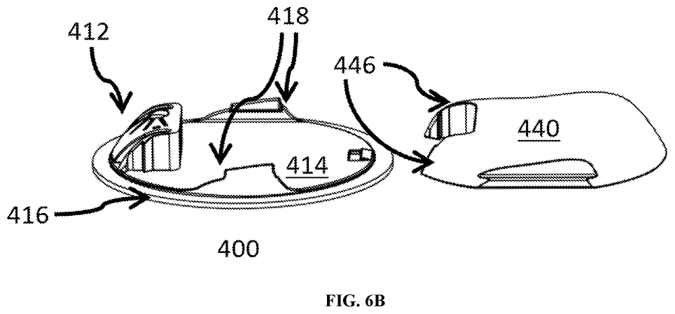

FIGS. 6A-6B provide perspective views of an example of a combined CGM infusion port with an external removable electronic module. FIG. 6A depicts an embodiment of the infusion device in which the electronic signal processing module is contained within a body that attaches to the skin-worn components of the device via two arms projecting from the signal processing module. The infusion device 400 includes a body 410 having an upper housing 412 and a base 414 that is attached to an adhesive patch 416, a cannula 420 that projects downward from the body, an access port 430 on the top surface of the cannula housing, an inserter port 462, and an electronic signal processing module 440 that interfaces with the cannula housing. An inserter port 462 allows an inserter needle to be placed through the housing to surround the cannula 420. An access port 430 permits a user (e.g., a subject, a patient, a physician, a nurse, a clinician, or a caretaker of the subject) to reversibly attach a fluid delivery device (e.g., a syringe, a pen needle, or an insulin pump) to the subject. This fluid may be a drug, diagnostic agent, or other liquid that is desired for subcutaneous infusion.

As shown in FIG. 6B, the electronic signal processing module 440 is removable and is shown separated from infusion device body 410. The electronic signal processing module 440 is reversibly attached to the base 414 and the upper housing 412 by a set of arms 446 that make contact with the vertical side edges of the upper housing 412. A set of guides 418 may be present on either side of the electronic signal processing module 440 in order to help retain electronic signal processing module 440. In some embodiments, the infusion components, such as the cannula 420, are disposable and have a use life limited to 3 or more days. By configuring the electronic signal processing module 440 such that it may be removed, it can be reused repeatedly, thereby reducing the recurring cost of the system. However, in other embodiments, the transmitter is permanently affixed to the infusion device body and may be discarded with the infusion device.

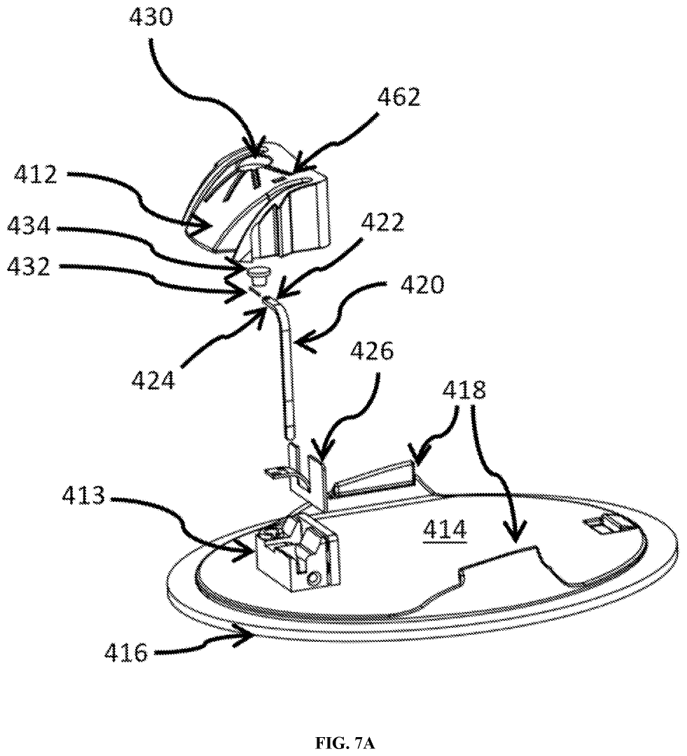

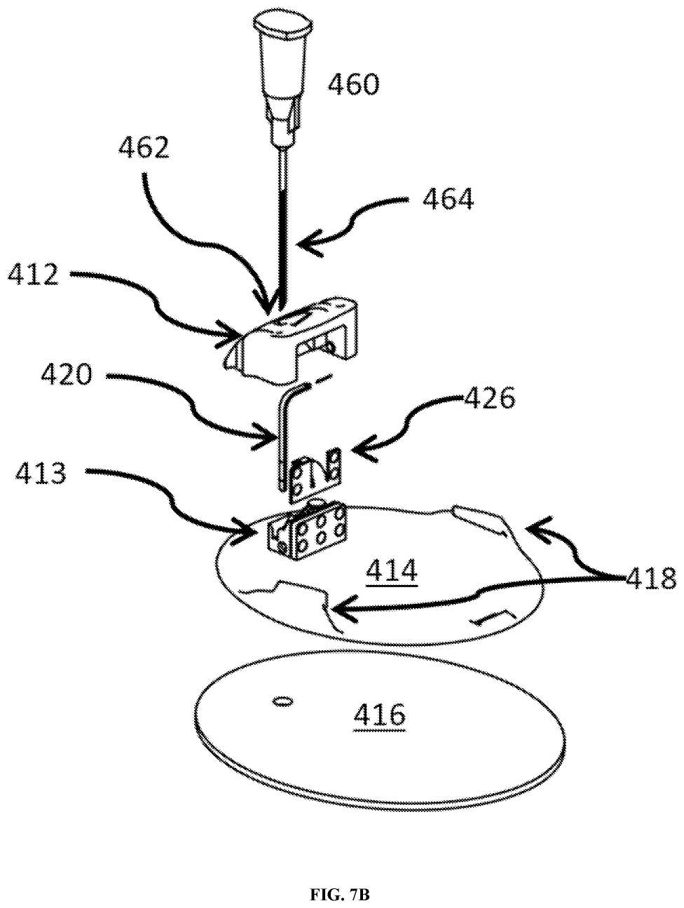

FIGS. 7A-7B provide exploded views of the combined CGM infusion port of FIGS. 6A-6B, including a view of an inserter needle (FIG. 7B). FIG. 7A depicts an exploded view of an embodiment of the infusion device prior to assembly in which the electronic signal processing module has been removed. The infusion device 400 includes a body 410 having an upper housing 412 and a base 414, an adhesive patch 416, a fluid path coupling needle 432, a septum 434, an access port 430 on the top surface of the cannula housing, and a sensing cannula 420 projecting downward from the body following assembly. A septum 434 may be made of self-sealing silicone or other elastomeric material, and serves to permit attachment to a fluid source when it is pierced. An electronic interconnect circuit 426 is inserted into a sensor housing 413, and makes contact and electrical connection at its proximal end to a set of contacts 422 and 424 on the top and bottom faces of the proximal end of the sensing cannula 420. A circuit 426 also makes contact at its distal end with the contacts of the electronic signal processing module 440 via pogo pins, conductive rubber buttons, or other interconnect device on the vertical face of the electronic signal processing module 440. The base 414 also has a set of retention arms 418 for holding the electronic signal processing module 440. Although these are shown as independent arms, they may connect to enclose the transmitter.

FIG. 7B depicts an exploded view of an embodiment of the infusion device configured with an insertion device used to place the sensing cannula into the subcutaneous tissue of a subject. The base 414 is affixed to an adhesive patch 416 used to affix the device to the skin, and the sensor housing 413 is attached to the top face of the base 414. The sensing cannula 420 is held by the upper housing 412 and the sensor housing 413, and is held in physical and electrical contact with the flex circuit 426. The insertion device 460 is placed through the insertion device guide channel 462 in the upper housing 412, which may contain a self-sealing septum to seal the opening remaining following removal of the insertion device. The insertion device may include a rigid, hollow structure 464 comprising a rigid material such as stainless steel. The hollow structure 464 is coaxial with and encloses the sensing cannula 420 after assembly. In some embodiments, the hollow structure 464 is used to pierce the skin of the subject for placement of the sensing cannula 420 into the subcutaneous compartment. An insertion device 460 may then be withdrawn through an opening 462, leaving the sensing cannula 420 positioned within the tissue of the subject. The embodiment here is shown essentially perpendicular. In other embodiments, the sensing cannula 420 may be positioned at an angle, such that the sensing cannula 420 may form an angle of between about 30 degrees to about 45 degrees (e.g., about 30 degrees, about 31 degrees, about 32 degrees, about 33 degrees, about 34 degrees, about 35 degrees, about 36 degrees, about 37 degrees, about 38 degrees, about 39 degrees, about 40 degrees, about 41 degrees, about 42 degrees, about 43 degrees, about 44 degrees, or about 45 degrees) between the base of the device 414 and the plane of the skin surface. The sensing cannula 420 may also be inserted at an extremely shallow angle (e.g., about 1 degree, about 2 degrees, about 3 degrees, about 4 degrees, about 5 degrees, about 6 degrees, about 7 degrees, about 8 degrees, about 9 degrees, about 10 degrees, about 11 degrees, about 12 degrees, about 13 degrees, about 14 degrees, about 15 degrees, about 16 degrees, about 17 degrees, about 18 degrees, about 19 degrees, about 20 degrees, about 21 degrees, about 22 degrees, about 23 degrees, about 24 degrees, about 25 degrees, about 26 degrees, about 27 degrees, about 28 degrees, or about 29 degrees), slightly below the skin surface, as in the case of a microneedle.

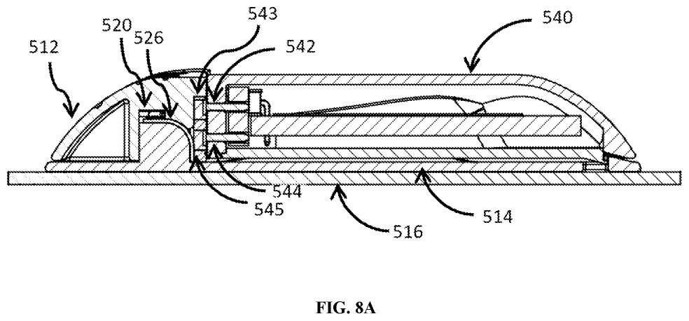

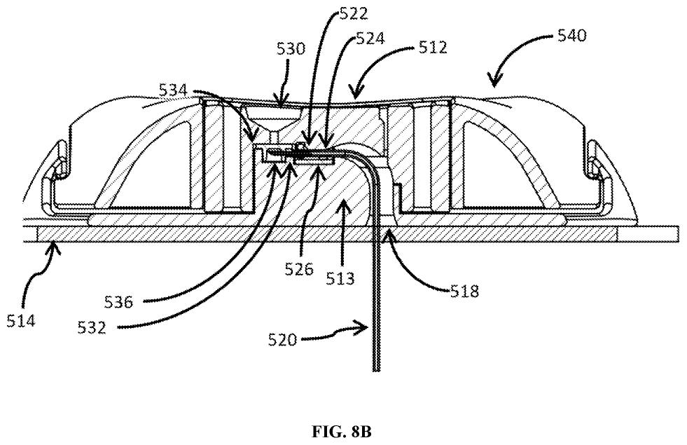

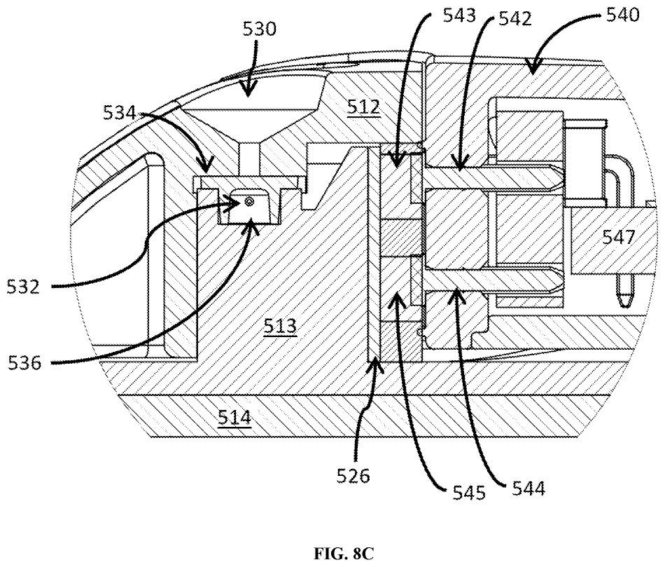

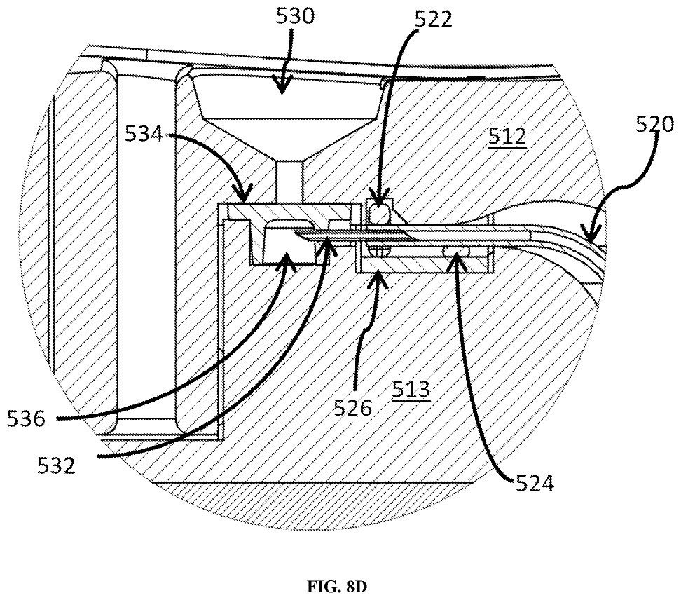

FIGS. 8A-8D provide cross-sectional views of an example of a combined CGM infusion port, including views of the interconnect detail. FIGS. 8A-8B depict cross-sections of an embodiment of an infusion device in which the electronic signal processing module is attached, transiently or permanently, to a body that contains the skin-worn components of the device. FIGS. 8C-8D depict greater detail of the electrical and fluid path connections to the combined sensing cannula. An electronic signal processing module 540 is shown attached to the body 510. Electrical connections to the sensing cannula from the signal processing module are provided via a set of electrical contacts 542 and 544 on the module, electrically connected with a set of contacts on interconnect circuit 526 through a set of conductive interface material 543 and 545. This material may comprise conductive rubber, a zebra connector, or similar selectively conductive compressible material. Although two contacts are shown, there may be only a single contact, or more than two contacts to carry additional signals. An interconnect circuit 526, which may be a flex circuit, is in turn in electrical contact with a set of sensor contacts 522 and 524 at the proximal end of the sensing cannula 520. This contact may be established using various suitable electrical connection materials such as solder or conductive epoxy. The connection may also be coated with a waterproof epoxy adhesive or other encapsulant to prevent moisture intrusion. A fluid connection to the proximal end of the combined analyte sensor and infusion cannula 520 is established via a connecting tube 532 held in a sensor housing 513 that permits fluid to flow from an adjacent needle cavity 536 formed by a sensor housing 513 and a self-sealing septum 534. The sensing cannula 520 exits through the base 514 through an opening 518. Access to a needle cavity 536 is provided through an opening 530 in a housing 512 and through penetration of the self-sealing septum 534 by the fluid delivery device.

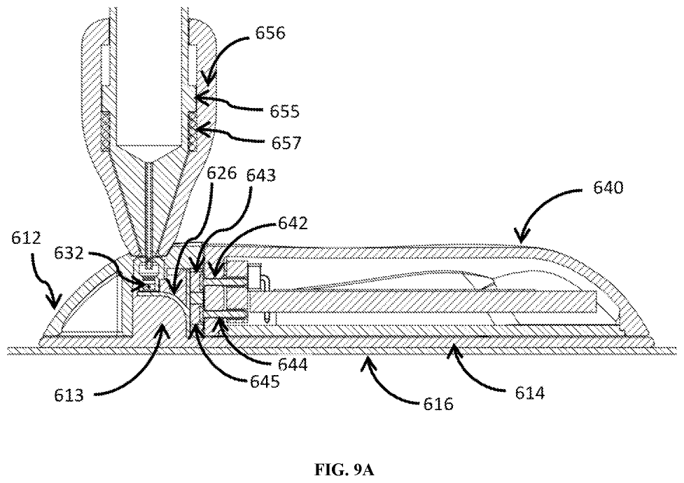

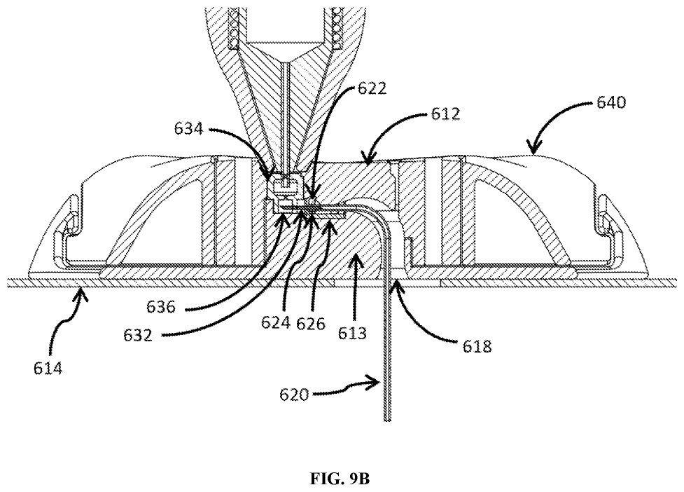

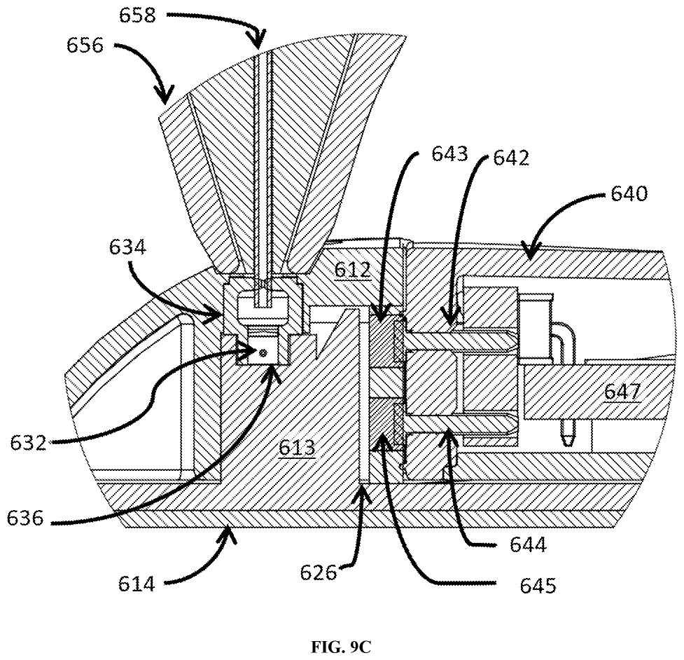

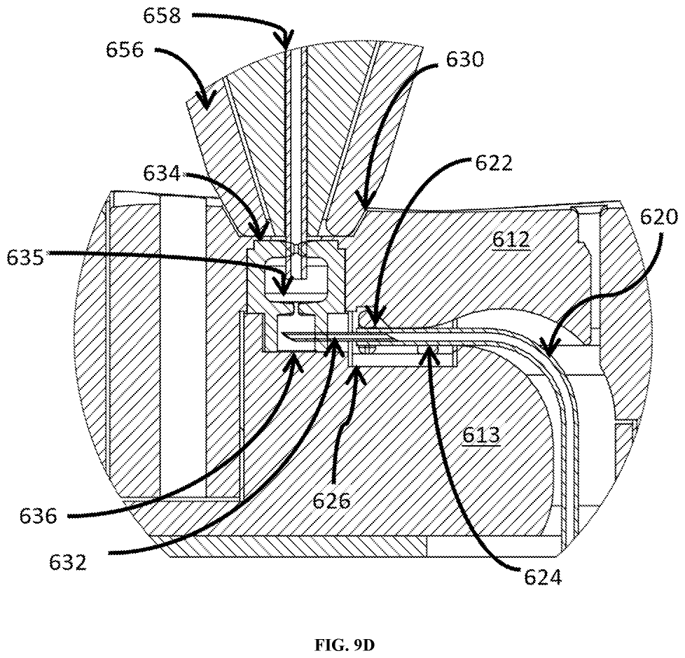

FIGS. 9A-9D provide cross-sectional views of an example of a combined CGM infusion port in contact with a needle-free insulin pen tip. FIGS. 9A-9B depict cross-sections of an embodiment of an infusion device in which the fluid path is configured to interface or couple (e.g., mate) with a drug delivery device. FIGS. 9C-9D depict greater detail of the electrical and fluid path connections to the combined sensing cannula. An electronic signal processing module 640 is shown attached to the body 610. A set of electrical connections from the sensing cannula to the PC board 647 within the signal processing module are established via a set of electrical contacts 642 and 644 on the module, electrically connected with a set of contacts on interconnect circuit 626 via a set of conductive interface material 643 and 645. This material may comprise conductive rubber, a zebra connector, or similar selectively conductive compressible material. Although two contacts are shown, there may be only a single contact, or more than two contacts to carry additional signals. An interconnect circuit 626, which may be a flex circuit, is in turn in electrical communication with a set of sensor contacts 622 and 624 at the proximal end of the sensing cannula 620. This contact, depicted in the cross-sectional view of FIG. 9D as balls on the sensor surface, may comprise electrical connection materials such as solder, conductive epoxy, or carbon paste. Contact may be made on both the upper and lower face if the set of contacts 622 and 624 are on opposing sides (as depicted), or with both contacts on the lower face if the sensor is configured with both contacts on the same face. The connections may also be coated with a waterproof epoxy adhesive or other encapsulant to prevent moisture intrusion. A fluid connection to the proximal end of the combined analyte sensor and infusion cannula 620 is established via a connecting tube 632 held in a sensor housing 613 that permits fluid to flow from a vestibule 636 formed by the sensor housing 613 and a septum 634. The septum 634 has a pre-formed central hole that is normally closed, but allows a blunt tube 658 contained within mating pen tip 656 to be pressed through it. The septum 634 may also have a check valve 635 in the fluid path, such as a ball or cross-slit valve, which serves to prevent retrograde flow of fluid (e.g., the drug or interstitial fluid) when the pen tip is removed. This has the advantage of preventing the attached pen tip tube 658 from being a biohazard. The housing 613 may also have an alignment feature 631 to guide the pen tip 656 to proper alignment during mating. The pen tip 656 may slide across the pen housing 655 through the action of a compressible spring 657. The sensing cannula 620 exits the base 614 attached to the skin of the subject via an adhesive patch 616 through an opening 618.

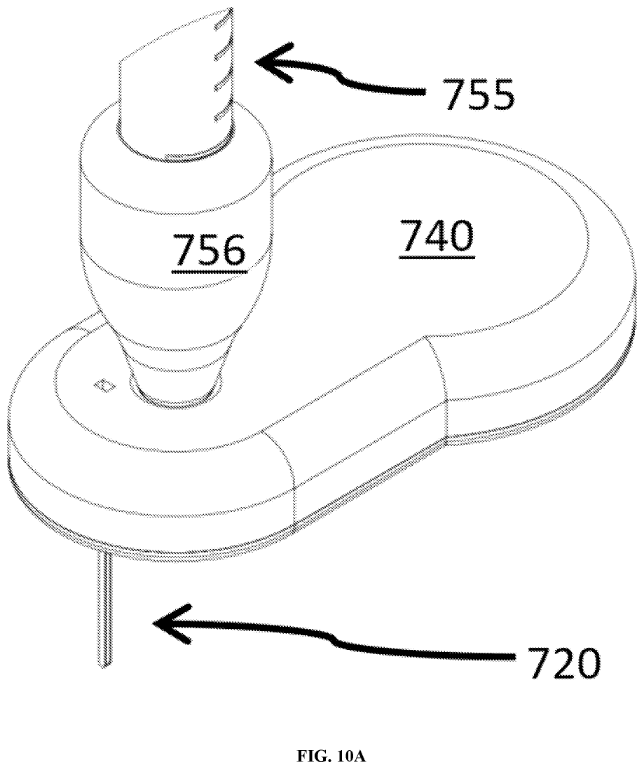

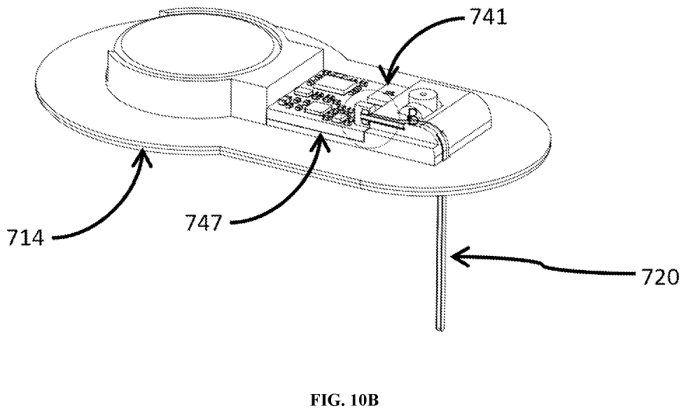

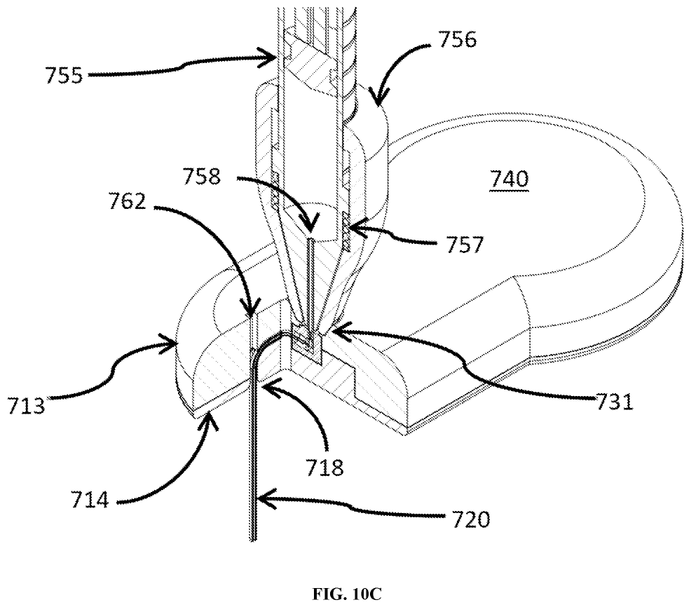

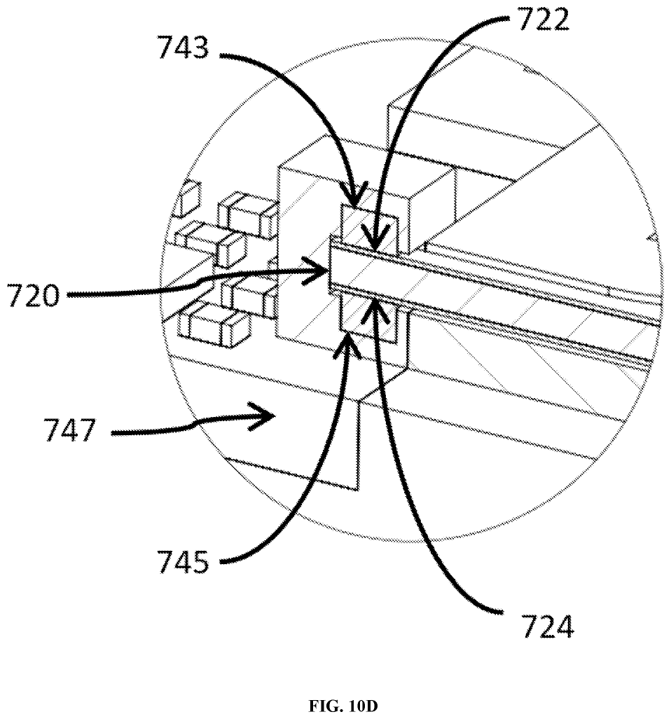

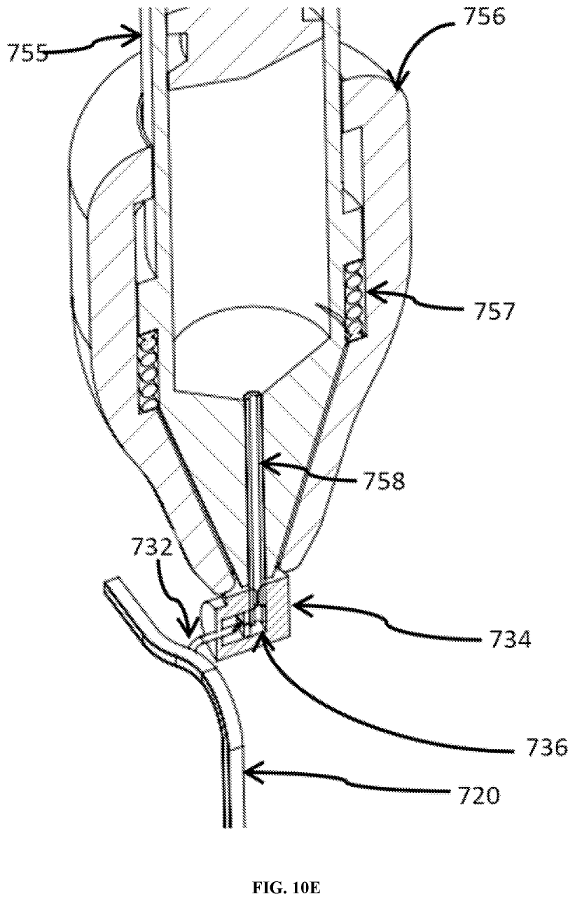

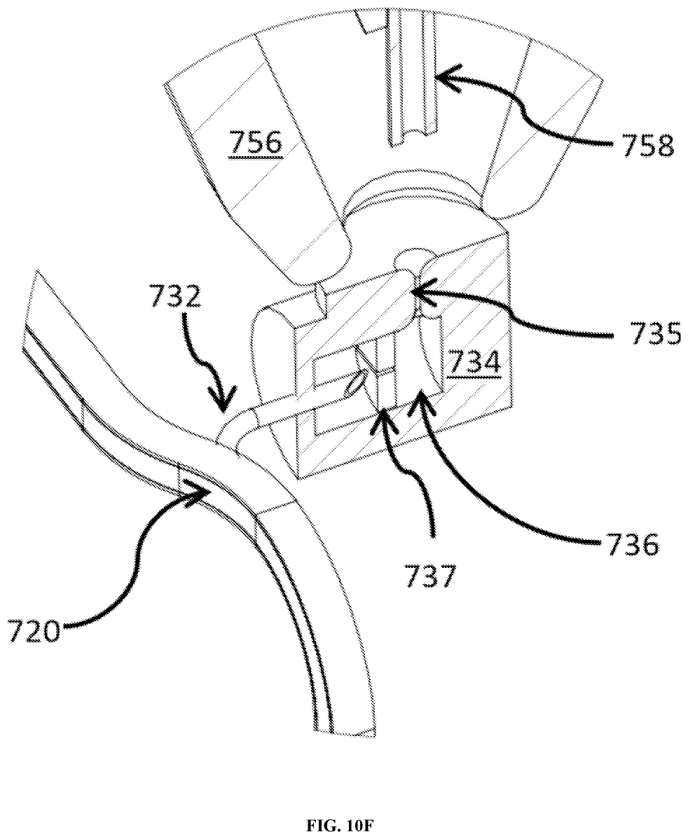

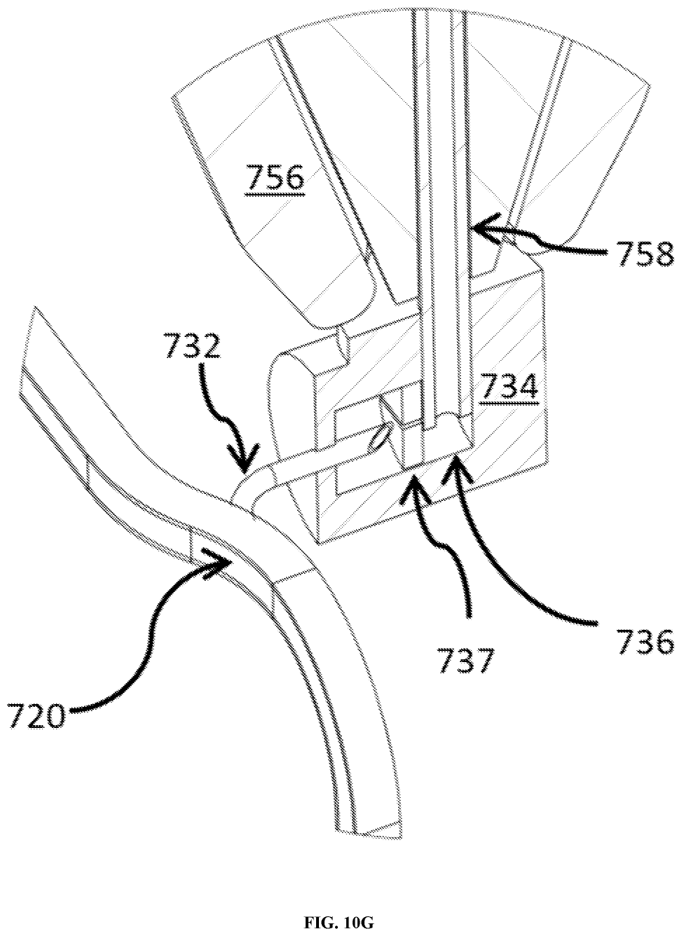

FIGS. 10A-10G provide views of an example of a disposable CGM infusion port in contact with a pen having a needle-free insulin pen tip. FIGS. 10A-10B depict perspective views of an embodiment of an infusion device in which the fluid path is configured to mate with a proprietary drug delivery device. FIG. 10C shows a cutaway view to display fluid path detail, and FIG. 10D includes greater detail of the electrical connections to the combined sensing cannula. An electronic signal processing module 740 is shown configured for a disposable application in which the signal processing electronic module 741 and the sensing cannula 720 are housed within a single continuous element supported on a housing base 714. A proprietary pen tip 756 is shown engaged with complementary alignment features in the housing of 740. The housing 713 may also have an alignment feature 731 to guide the pen tip 756 to proper alignment during mating. The pen tip 756 may slide across the pen housing 755 through the action of a compressible spring 757. The fluid is shown being delivered from the internal cavity of a pen 755 through the hollow tube 758 and into the infusion device. The fluid exits through sensing cannula 720, which extends through the base 714 via an opening 718. A channel 762 allows for the temporary placement of an inserter needle. Greater fluid path detail is depicted in FIGS. 10E-10G. FIGS. 10E-10G provide sectional views of the disposable CGM infusion port in contact with a pen having a needle-free insulin pen tip, including a sectional view with the pen tip attached (FIG. 10E), detail of the fluid path section with the pen tip disengaged from the fluid path (FIG. 10F), and details of the fluid path section with the pen tip engaged with the fluid path (FIG. 10G). A set of electrical connections to the sensing cannula 720 from the signal processing electronics 741 are provided via a set of electrical contacts 722 and 724 on the sensor surface contacting a socket having a set of contacts 743 and 745. This socket conveys signal currents onto PC board 747 containing the electronic signal processing electronic module. The socket contacts may comprise a metal spring or conductive rubber, or a zebra connector or similar selectively conductive compressible material. Although two contacts are shown, there may be only a single contact, or more than two contacts to carry additional signals. This contact may also comprise electrical connection materials such as solder or conductive epoxy. The connection may also be coated with a waterproof epoxy adhesive or other encapsulant to prevent moisture intrusion.

FIG. 10E-10G have been sectioned to show various internal features of the devices. FIG. 10F shows the pen tip 756 in contact but with fluid path tube 758 withdrawn, and FIG. 10G shows the same pen tip with fluid path tube 758 fully inserted. As shown in these section views of FIGS. 10E-10G, a fluid connection to the proximal end of the sensing cannula 720 is established via a connecting tube 732 held in a sensor housing 713 that permits fluid to flow from a vestibule 736 within a fluid path connector 734. The fluid path connector 734 may comprise an elastomeric component created by molding a material such as silicone or a rubber, such as butyl rubber or ethylene propylene diene monomer (EPDM) rubber. It has a pre-formed central hole 735 that is normally closed, but allows a blunt tube 758 contained within mating pen tip 756 to be pressed through it. The fluid path connector may also have a check valve 737 in the fluid path, such as a ball or cross-slit valve, which serves to prevent retrograde flow of fluid (e.g., the drug or interstitial fluid) when the pen tip is removed. This has the advantage of preventing the attached pen tip tube 758 from being a biohazard.

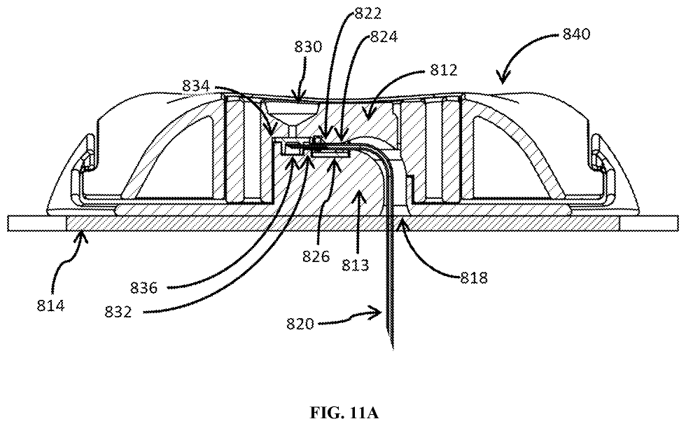

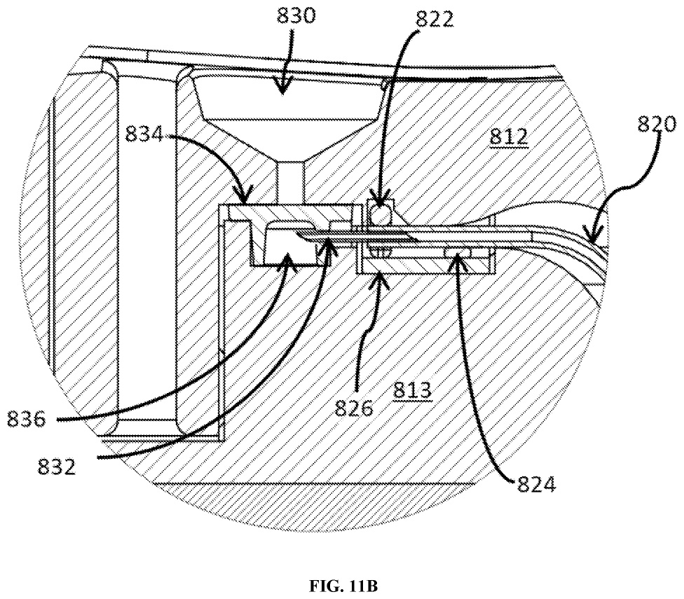

FIGS. 11A-11B provide views of an example of a combined CGM infusion port with a rigid sensor, including a front section view (FIG. 11A) and a front section view showing a fluid path and electrical contact detail (FIG. 11B). These figures depict a side cross-section of an embodiment of the infusion device in which the electronic signal processing module is attached, transiently or permanently, to a body that contains the skin-worn components of the device and a sensing cannula configured to be inserted without the aid of an inserter needle. An electronic signal processing module 840 is shown attached to the body 810. An interconnect circuit 826, which may be a flex circuit, is in electrical contact with a set of sensor contacts 822 and 824 at the proximal end of the sensing cannula 820. These sensor contacts may be on the same face of the cannula, or on opposite faces. This contact may comprise electrical connection materials such as solder or conductive epoxy, and may be encapsulated by a waterproof material such as epoxy or other encapsulant. A fluid connection to the proximal end of the sensing cannula 820 is established via a connecting tube 832 held in a sensor housing 813 that permits fluid to flow from an adjacent needle cavity 836 formed by the sensor housing 813 and a self-sealing septum 834. The sensing cannula 820 exits through a base 814 through an opening 818, and is configured with a sharpened tip and sufficient rigidity to penetrate the skin of the subject without the need for a separate inserter needle. Access to a needle cavity 836 is provided through an opening 830 in an upper housing 812 and through penetration of a self-sealing septum 834 by the fluid delivery device.

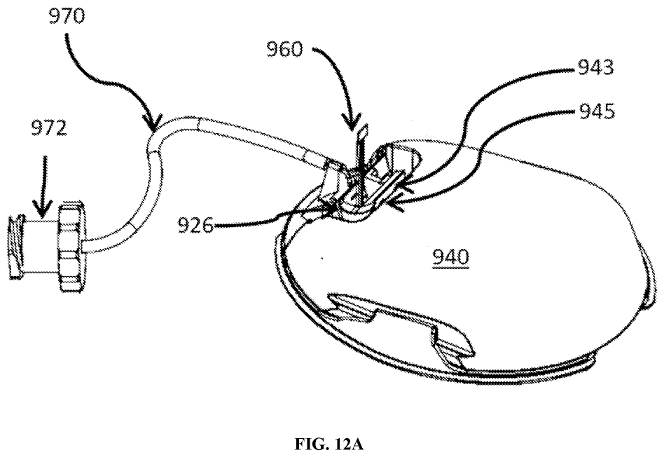

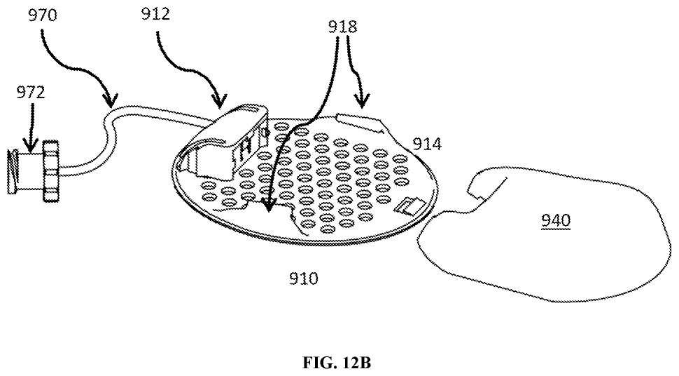

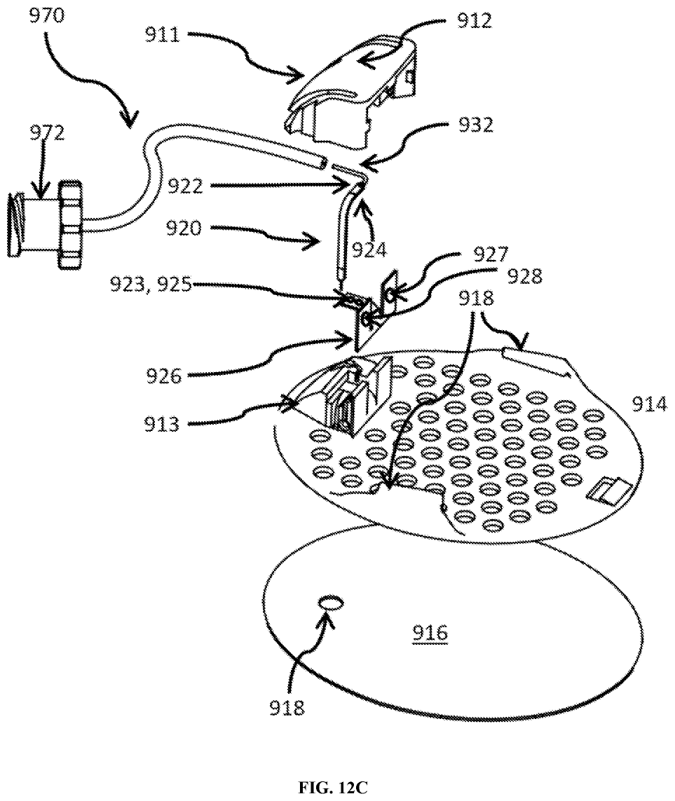

FIGS. 12A-12C provide perspective views (FIGS. 12A-12B) and an exploded view (FIG. 12C) of an example of a combined CGM infusion port configured for attachment to an insulin pump or gravity-fed source of medication. FIGS. 12A-12B depict perspective views of an embodiment of an infusion device configured to co-locate electrical and fluid connections to a sensing cannula, further configured for use with an insulin pump or gravity-fed fluid source. FIG. 12C depicts an exploded view of an embodiment of an infusion device configured to co-locate electrical and fluid connections to a unified analyte sensor and fluid delivery cannula 920, further configured for use with an insulin pump or gravity-fed fluid source. A body 910 is shown separated from an electronic signal processing module 940. In an embodiment, an infusion tubing 970 projects from an opening 911 formed by an upper housing 912 and a sensor housing 913. The infusion tubing 970 has an in-line connector 972 permitting temporary attachment to a mating fluid pump connector, which is connected to a source of a therapeutic liquid such as a drug delivery pump or gravity-fed source. In some embodiments, the infusion tubing 970 is attached to the body 910 via a connector at the body terminus (e.g., having one or more cantilever snap joints that allow reversible attachment of the tubing to the body). A connection of the fluid source to the sensing cannula 920 is established via a fluid path coupler 932 inserted into the infusion tubing 970. The sensing cannula 920 exits through the base 914 and an adhesive patch 916 via an opening 918. A flexible circuit 926 establishes electrical contact with a set of contacts 922 and 924 on the proximal end of the sensing cannula 920 inside of the cap 912. Electrical contacts 923 and 925 on the proximal end of the flexible circuit 926, are held in contact with a set of contacts 922 and 924 at the proximal end of the sensing cannula 920. An electrical connection to the sensor electronic module 940 is provided by a set of elastomeric electrical contacts 943 and 945 exposed to contact with the sensor electronic module 940. These contacts establish electrical connection with a set of contacts 927 and 928 on the flexible circuit 926 via their opposite face. A set of retention arms 918 are provided on the base 914 for temporary attachment of the sensor electronic module 940. FIG. 12A shows an inserter needle 960 used to insert the cannula into the tissue of a subject (e.g., a human, an animal, or a mammal).

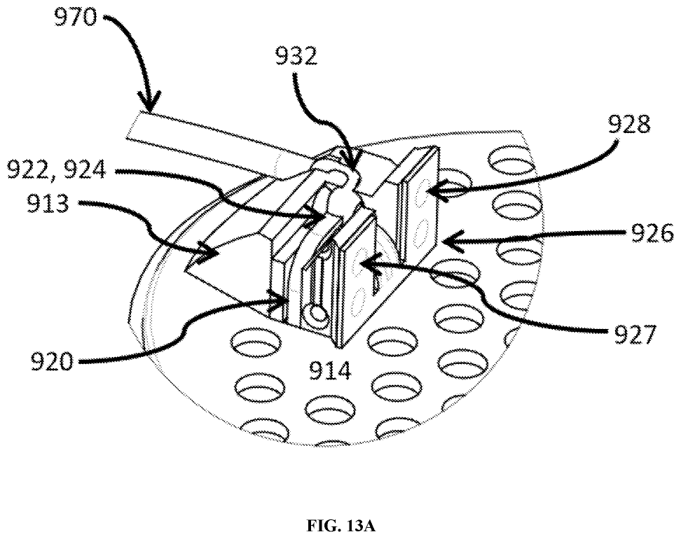

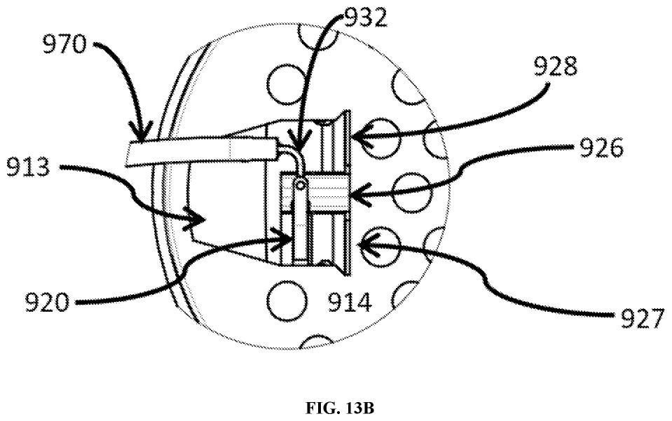

FIGS. 13A-13B provide a perspective view (FIG. 13A) and a top sectional view (FIG. 13B) of an example of a combined CGM infusion port configured for attachment to an insulin pump or gravity-fed source of medication with electronic module removed, showing a fluid path and electrical interconnect detail. These detailed views relate to an embodiment of an infusion device configured to co-locate electrical and fluid connections to a sensing cannula, further configured for use with an insulin pump or gravity-fed fluid source. In an embodiment, the infusion tubing 970 projects from the opening 911 in the sensor housing 913. The infusion tubing 970 comprises an in-line connector 972, which permits temporary attachment to a mating fluid pump connector 974, which provides a fluid connection to a therapeutic fluid source (e.g., a drug delivery pump or gravity-fed source). Connection of the fluid source to the sensing cannula 920 is established via a fluid path coupler 932 inserted into the infusion tubing 970, which passes through the opening 911 in the cap 912. The sensing cannula 920 exits through the base 914 via the opening 918. An electrical connection to the sensing cannula 920 is established via a set of electrical contacts 923 and 925 on the flexible circuit 926, held in contact with the set of contacts 922 and 924 at the proximal end of the sensing cannula 920. An electrical connection to the sensor electronic module 940 is established by a set of elastomeric electrical contacts on the module, which are in electrical connection with a set of contacts 927 and 928 on the flexible circuit 926. A set of retention arms 918 are provided on the base 914 for temporary attachment of the sensor electronic module 940.

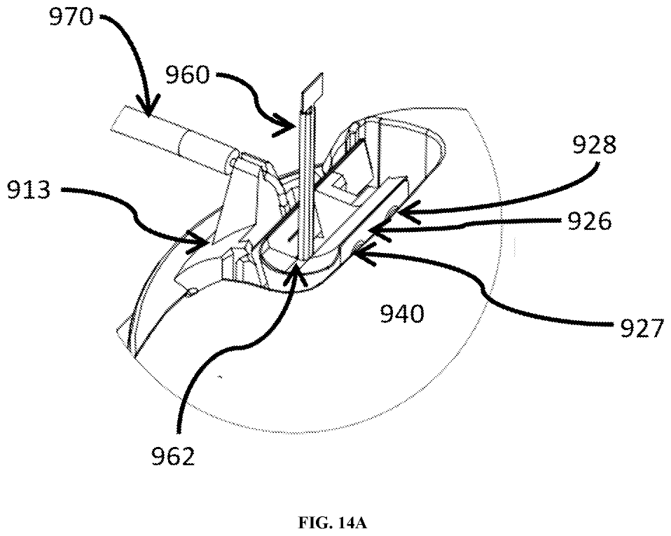

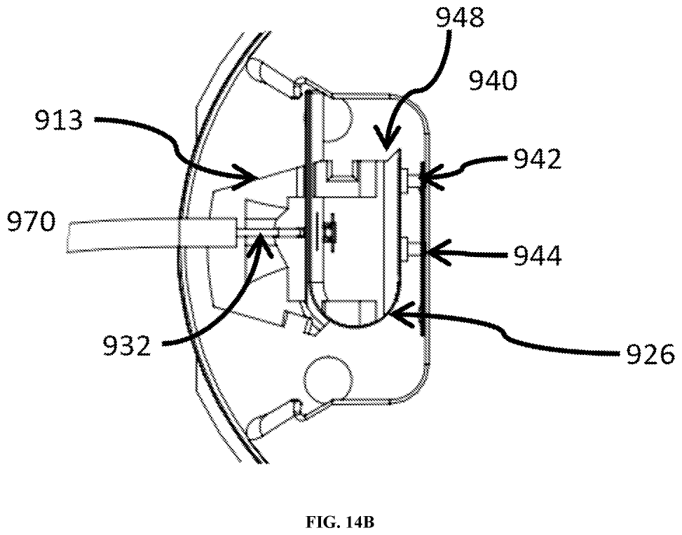

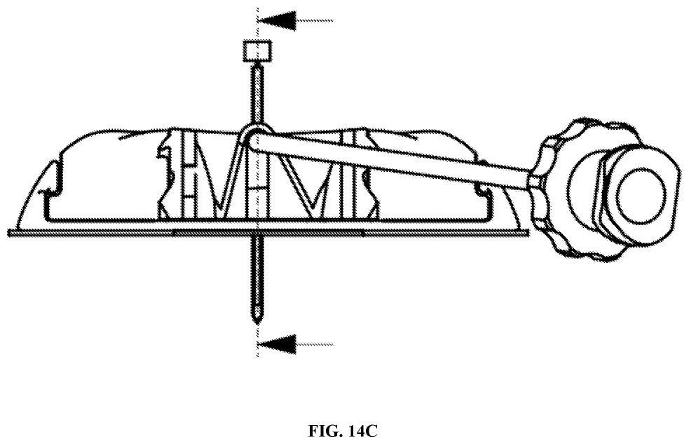

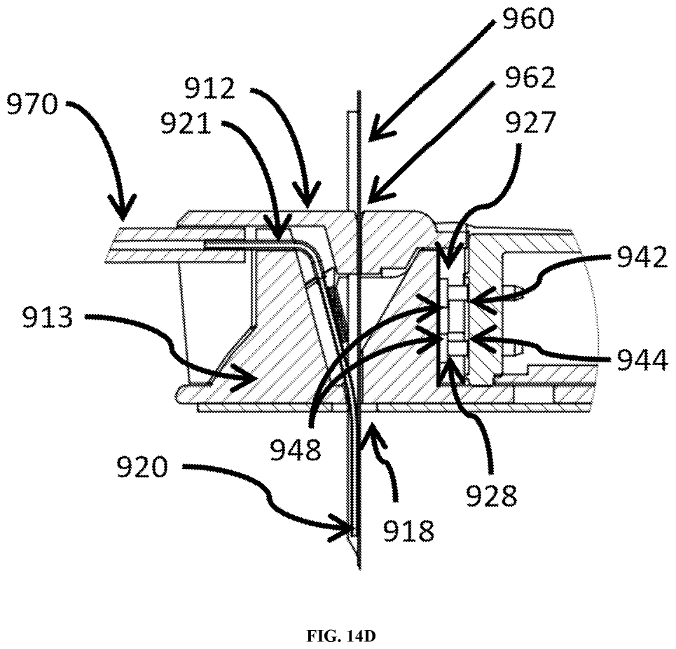

FIGS. 14A-14D provide a perspective view (FIG. 14A), a top sectional view (FIG. 14B), a front sectional view (FIG. 14C), and a side sectional view (FIG. 14D) of an example of a combined CGM infusion port configured for attachment to an insulin pump or gravity-fed source of medication with a rigid inserter needle or trocar. FIGS. 14-14B show the interconnect to electronics. FIGS. 14-14D show the tubed infusion set. FIGS. 14A-14B depict detailed views of an embodiment of an infusion device configured to co-locate electrical and fluid connections to a sensing cannula 920, further configured for use with an insulin pump or gravity-fed fluid source, with an insertion needle configured for placement of the sensing cannula 920 into the tissue. In an embodiment, the infusion tubing 970 projects from the opening 911 in the sensor housing 913. The inserter 960 is a long, needle-like open metal piece having a square cross section with three sides used to enclose the sensing cannula 920. The inserter 960 is placed through the inserter port 962. An electrical connection to the sensor electronic module 940 is established by the set of electrical contacts 927 and 928 on the flexible circuit 926. A compressible material 948 is placed behind the set of contacts 927 and 928 to accommodate compression by the set of contact pins 942 and 944 on the sensor electronic module 940.

FIGS. 14C-14D depict detailed views of an embodiment of an infusion device configured to co-locate electrical and fluid connections to a sensing cannula 920, further configured for use with an insulin pump or gravity-fed fluid source, in which the sensor fluid path is provided by a rigid tube. In an embodiment, the infusion tubing 970 projects from the opening 911 in the sensor housing 913. The upper housing 912 encloses and secures the elements below it. The sensing cannula 920 has a fluid path comprising a preformed tube 921 that is inserted directly into the infusion tubing 970. The connection may be sealed with a biocompatible adhesive, or bonded directly to the infusion tubing 970 using an adhesive or thermal bonding techniques. An electrical connection to the sensor electronic module 940 is established by the set of electrical contacts 927 and 928 on the flexible circuit 926. The compressible material 948 is placed behind the set of contacts 927 and 928 to accommodate compression by the set of contact pins 942 and 944 on the sensor electronic module 940.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-100. (canceled)

101. A device configured to perform simultaneous sensing of a concentration of an analyte and administration of a therapeutic fluid, comprising:
a body comprising an upper housing, a lower housing, and a bottom, skin-contacting base, wherein the upper housing comprises a port configured to reversibly attach to a fluid delivery device configured for delivery of a fluid via insertion of a needle, wherein the port comprises a self-sealing septum in contact with the lower housing thereby forming an internal cavity;
a sensing cannula comprising a proximal end, a distal end, an external surface, an internal lumen, at least one hollow channel within the internal lumen extending from the proximal end of the sensing cannula to the distal end of the sensing cannula configured for the administration of the therapeutic fluid, at least one indicating electrode on the external surface configured to sense the concentration of the analyte, and a conductor on the external surface extending from the proximal end of the sensing cannula to the at least one indicating electrode, wherein the proximal end of the sensing cannula is retained within the body, and wherein the distal end of the sensing cannula extends from the skin-contacting base; and
a channel within the body in fluid communication with the internal cavity formed by the self-sealing septum and the proximal end of the combined sensing cannula.

102. The device of claim 101, wherein the upper housing comprises a top face comprising the port.

103. The device of claim 101, wherein the port comprises a visible opening comprising the self-sealing septum.

104. The device of claim 101, further comprising a signal processing module configured to receive an electrical current from the sensing cannula.

105. The device of claim 104, wherein the signal processing module is configured to provide an electrical potential to the sensing cannula.

106. The device of claim 105, wherein the signal processing module comprises a second body comprising an upper face, a lower face, and a vertical surface between the upper face and the lower face.

107. The device of claim 106, wherein the vertical surface provides the electrical potential to the sensing cannula and receives the electrical current from the sensing cannula via a set of electrical contacts on the vertical surface.

108. The device of claim 107, wherein the second body comprises a set of arms in contact with the upper housing, and wherein the lower face is in contact with the skin-contacting base.

109. The device of claim 104, further comprising an interface circuit configured to convey current signals from the sensing cannula to the signal processing module.

110. The device of claim 109, wherein the interface circuit comprises a proximal end and a distal end.

111. The device of claim 110, wherein the interface circuit comprises one or more conductors configured to convey the current signals from the sensing cannula to the signal processing module.

112. The device of claim 101, wherein the fluid delivery device comprises a syringe.

113. The device of claim 101, wherein the fluid delivery device comprises a pen.

114. The device of claim 101, wherein the at least one indicating electrode comprises an enzyme layer overlaying a conductive surface.

115. The device of claim 114, wherein the enzyme layer is covered with a semi-permeable membrane.

116. The device of claim 114, wherein the enzyme layer comprises glucose oxidase or glucose dehydrogenase.

117. The device of claim 114, wherein the enzyme layer comprises an osmium-based redox mediator.

118. The device of claim 117, wherein the osmium-based redox mediator comprises osmium dimethyl bipyridine.

119. The device of claim 114, wherein the enzyme layer comprises polyvinylimidazole.

120. The device of claim 101, wherein the sensing cannula comprises a reference electrode comprising silver/silver chloride (Ag/AgCl).

121. The device of claim 101, wherein the signal processing module provides a bias potential to the sensing cannula of less than 250 millivolts (mV) relative to a reference potential.

122. The device of claim 101, wherein the upper housing and the lower housing are configured to receive a hollow inserter needle partially enclosing the sensing cannula for insertion into a skin surface of a mammal.

123. The device of claim 101, wherein the sensing cannula comprises a stiffness sufficient for insertion into a skin surface of a mammal without using an inserter needle.

124. The device of claim 101, wherein the skin-contacting base comprises an adhesive surface configured to attach to a skin surface of a subject.

125. The device of claim 101, wherein the analyte is selected from the group consisting of: oxygen, glucose, lactate, a drug metabolite, and a pathogen.

126. The device of claim 125, wherein the analyte is glucose.

127. The device of claim 101, wherein the therapeutic fluid is selected from the group consisting of: an insulin or insulin analog formulation, glatiramer acetate, heparin, human menopausal gonadotropin, vitamins, and minerals.

128. The device of claim 127, wherein the therapeutic fluid is the insulin or the insulin analog formulation.

129. The device of claim 128, wherein the insulin or the insulin analog formulation comprises an excipient comprising a phenol or cresol.

130. A device configured to perform simultaneous sensing of a concentration of an analyte and administration of a therapeutic fluid, comprising:
a body comprising an upper housing, a lower housing, a bottom, skin-contacting base, and an infusion tubing extending outward from the body configured to connect to a source of the therapeutic fluid;
a sensing cannula comprising a proximal end, a distal end, an external surface, an internal lumen, at least one hollow channel within the internal lumen extending from the proximal end of the sensing cannula to the distal end of the sensing cannula configured for the administration of the therapeutic fluid, at least one indicating electrode on the external surface configured to sense the concentration of the analyte, and a conductor on the external surface extending from the proximal end of the sensing cannula to the at least one indicating electrode, wherein the proximal end of the sensing cannula is retained within the body, and wherein the distal end of the sensing cannula extends from the skin-contacting base; and
a channel within the body in fluid communication with the internal cavity formed by the self-sealing septum and the proximal end of the combined sensing cannula.
